(19) United States
(12) Patent Application Publication
(10) Pub. No.: US 2021/0187085 A1
(43) Pub. Date: Jun. 24, 2021

(54) PHOSPHOLIPASE A2 RECEPTOR ANTIGENS AND THEIR MEDICAL USE

(71) Applicant: The University of Manchester, Manchester (GB)

(72) Inventors: Paul BRENCHLEY, Manchester (GB); Thomas JOWITT, Manchester (GB); Maryline FRESQUET, Manchester (GB); Rachel LENNON, Manchester (GB); Sam RHODEN, Manchester (GB)

(73) Assignee: The University of Manchester, Manchester (GB)

(21) Appl. No.: 16/757,733

(22) PCT Filed: Oct. 23, 2018
(86) PCT No.: PCT/GB2018/053059

§ 371 Date: Apr. 20, 2020

(30) Foreign Application Priority Data
Oct. 23, 2017 (GB) 1717345.1

(51) Int. Cl. A61K 39/00; C07K 14/705; A61P 13/12

(52) U.S. Cl. CPC A61K 39/0008; A61P 13/12; C07K 14/7056

(57) ABSTRACT

The present invention relates to a non-naturally occurring peptide comprising amino acid sequence S-V-L-T-X1-E-N-X2 (SEQ ID NO: 1), wherein X1 is any amino acid and X2 is any amino acid, which may optionally be linked to an amino acid sequence X3-I-X4-X5-E-X6 (SEQ ID NO: 5), wherein, X3, X4, X5 and X6 each denote any amino acid. The present invention further relates to the use of such peptides as a medicament such as for the prevention and treatment of kidney disease and in apheresis methods.

### FIELD OF THE INVENTION

The present invention relates to non-naturally occurring peptides that are able to bind to anti-PLA2R antibodies, and also to such peptides for use in the prevention or treatment of kidney disease. The invention also relates to pharmaceutical compositions comprising a peptide and a pharmaceutically acceptable carrier. The invention relates to methods of preventing or treating kidney disease in a subject by providing a therapeutically effective amount of a peptide to a subject in need of such prevention or treatment, and to devices for extracorporeal treatment of a patient's blood, as well as methods of determining levels of anti-PLA2R antibodies in a subject.

### BACKGROUND

Idiopathic membranous nephropathy (IMN) is a rare form of glomerulonephritis affecting 10-12 cases per million population. The significant discovery in 2009 that circulating antibodies to phospholipase A2 receptor 1 (PLA2R) are present in 70% of patients with IMN, identified the autoimmune nature of this pathology. Genetic evidence of the involvement of PLA2R in IMN came from the genome wide association study identifying PLA2R and DQA1 as genes accountable for the genetic susceptibility to IMN. Clinical confirmation that anti-PLA2R antibodies are relevant in IMN is evident from studies showing an association between high levels of anti-PLA2R and active disease, poor clinical outcome at 5 years and less chance of spontaneous remission.

In other autoimmune kidney diseases such as anti-GBM disease, which is characterized by anti-collagen IV (α3NC1) autoantibodies and ANCA vasculitis characterized by anti-MPO autoantibodies, the antigenic epitopes include both linear peptide sequences and 3D conformational structure. Knowledge of these antigen epitopes in these diseases has been important in understanding the pathological disease mechanisms and may help to classify patient subgroups and severity of disease.

We have previously disclosed naturally occurring peptides which are able to bind anti-PLA2 antibodies, as well as a 28mer modified peptide in which specific lysine residues are conserved—see WO 2015/185949. However, there is a need for non-naturally occurring peptides which have improved properties in terms of: solubility, binding of the autoantibody, broader spectrum of binding across a patient population etc.

Furthermore, there is a need for shorter peptides which are expected to be less immunogenic than PLA2R and for peptides with improved solubility for use as a peptide mimetic.

### SUMMARY OF THE INVENTION

In one aspect, the present invention relates to a non-naturally occurring peptide comprising amino acid sequence S-V-L-T-X1-E-N-X2 (SEQ ID NO: 1), wherein X1 is any amino acid and X2 is any amino acid.

Suitably, the peptide of the invention may be a variant peptide comprising the amino acid sequence SVLTEENC (SEQ ID NO: 2) or SVLTEENS (SEQ ID NO: 3).

Suitably, the peptide may comprise:
A C-terminal domain comprising the amino acid sequence S-V-L-T-X1-E-N-X2 (SEQ ID NO: 1)
An N-terminal domain comprising the amino acid sequence X3-I-X4-X5-E-X6 (SEQ ID NO: 5), wherein X3, X4, X5 and X6 each denote any amino acid.

Suitably, X3 may be V or P; X4 may be Q, D or E; X5 may be S, D or E and X6 may be S, D or E.

Suitably, the peptide may be a variant peptide comprising the amino acid sequence S-V-L-T-X1-E-N-X2-K (SEQ ID NO: 6). Suitably, the peptide may be a variant peptide comprising the amino acid sequence S-V-L-T-X1-E-N-C-K (SEQ ID NO: 7).

Suitably, the N-terminal domain of the peptide may comprise the amino acid sequence X3-1-X4-X5-E-X6-L-K (SEQ ID NO: 8).

Suitably, the peptide comprises a linker between an N-terminal and a C-terminal domain. Suitably, the linker may be selected from a group consisting of: a peptide linker, a synthetic linker, a combination of peptide and synthetic linker.

Suitably, the linker may separate the N-terminal and C-terminal domains by a distance greater than or equal to a linker consisting of 5 glycine residues.

Suitably, the linker may comprise 5 glycine residues or may be a synthetic linker comprising a PEG molecule. Suitably, the synthetic linker may further comprise a lysine residue.

Suitably, the total number of amino acid residues in the peptide may be less than or equal to 28 amino acid residues; or less than or equal to 19 amino acid residues.

Suitably, the peptide may be one in which X1 may be L or E and/or X2 may be C or S.

Suitably, the peptide may comprise an N-terminal domain which comprises a variant of the sequence VIQSES (SEQ ID NO: 9) such that one or more of the following substitutions are made: V to P, Q to D or E, optionally either or both S to D or E. For example, the N-terminal domain may comprise the sequence PIDDES (SEQ ID NO: 10) or PIESES (SEQ ID NO: 11).

Suitably, the peptide may comprise the amino acid sequence X3-I-X4-X5-E-X6-PEG-K-PEG-S-V-L-T-X1-E-N-X2 (SEQ ID NO: 12).

Suitably, the peptide may comprise PIESES-PEG-K-PEG-SVLTEENC (SEQ ID NO: 13); VIQSES-PEG-K-PEG-SVLTLENC (SEQ ID NO: 14); VIQSES-PEG-K-PEG-SVLTEENC (SEQ ID NO: 15); PIDDES-PEG-K-PEG-SVLTLENC (SEQ ID NO: 16); PIDDES-PEG-K-PEG-SVLTEENC (SEQ ID NO: 17).

Suitably, the peptide of the invention may be soluble.

In another aspect, the present invention provides a pharmaceutical composition comprising a peptide of the invention and a pharmaceutically acceptable carrier.

In a further aspect, the present invention provides a peptide of the invention or a pharmaceutical composition of the invention for use as a medicament.

Suitably, the peptide of the invention or the pharmaceutical composition of the invention may be for use in the prevention or treatment of kidney disease.

In another aspect, the present invention provides a method of preventing or treating kidney disease in a subject, the method comprising administering a therapeutically effective amount of a peptide of the invention to a subject in need thereof.

Suitably, the peptide may be provided in a pharmaceutical composition of the invention.

Suitably, the therapeutically effective amount of the peptide may be an amount of the peptide that provides a therapeutically effective inhibition of binding of anti-PLA2R antibodies to native PLA2R in the subject.

In a further aspect, the present invention provides the use of a peptide in accordance with the invention or a pharmaceutical composition of the invention in the manufacture of a medicament.

Suitably, the medicament may be for use in the prevention or treatment of kidney disease.

In another aspect, the present invention provides a device for extracorporeal treatment of a patient's blood, the device comprising:
a peptide of the invention; and
means for separating the peptide from blood.

Suitably, the means for separating the binding partner from blood may comprise a substrate of a material selected from the group consisting of: cellulose; cellulose derivatives; agarose; agarose derivatives; polysulphone; polysulphone derivatives; polyacrylamide; polyacrylamide derivatives; and nylon.

Suitably, the device may be in a form selected from the group consisting of: hollow fibre cassettes, membranes, and beads.

Suitably, the device may be a column.

In another aspect, the present invention provides a method of preventing or treating kidney disease in a subject, the method comprising:
1. contacting a volume of the subject's blood with at least 1 peptide of the invention, such that anti-PLA2R antibodies present in the subject's blood are able to bind to and be retained by the peptide; and
2. separating the peptide and bound anti-PLA2R antibody from the blood, to yield an antibody-depleted volume of blood.

Suitably, a batch of blood comprising one or more volumes of blood to be treated, may be removed from the subject, and the steps of contacting a volume of the blood with at least 1 peptide, and subsequent separation of the peptide and bound antibodies, may be completed to yield a batch comprising the volume, or volumes, of antibody-depleted blood.

Suitably, the steps of contacting the blood with the binding partner, and subsequent separation, may be carried out “in line”.

Suitably, the binding partner may be provided in an arrangement so that the patient's blood may flow over the binding partner, thus allowing it to bind anti-PLA2R antibodies in the blood.

Suitably, at least 1 peptide may be provided as part of a device of the invention.

Suitably, in all aspects referring to kidney disease, the kidney disease may be selected from the group consisting of: primary renal failure; membranous nephropathy, such as idiopathic membranous nephropathy or de novo membranous nephropathy; or recurrent membranous nephropathy in a transplant

In a further aspect, the present invention provides a method of determining levels of anti-PLA2R antibodies in a subject, the method comprising:
contacting at least 1 peptide of the invention with a sample of a body fluid from the subject, such that anti-PLA2R antibodies present in the subject's body fluid sample are able to bind to and be retained by the peptide; and
determining the amount of anti-PLA2R antibodies retained by the peptide.

Suitably, the method may be for use in diagnosing kidney disease in a subject, the method comprising:
determining the amount of anti-PLA2R antibodies in a body fluid sample from a subject in accordance with the invention; and
comparing the determined amount with a reference value, wherein if the determined amount of anti-PLA2R antibodies in the sample is equal to or greater than the reference value, this indicates that the subject has kidney disease.

Suitably, the subject may be one that is considered to be at risk of kidney disease.

Suitably, the methods may be carried out in vitro.

In a further aspect, the present invention provides a method for selecting a suitable regimen for the prevention or treatment of kidney disease, the method comprising:
determining the amount of anti-PLA2R antibodies in a body fluid sample from a subject in accordance with the invention; and
comparing the determined amount with a reference value,
   wherein if the determined amount of anti-PLA2R antibodies in the sample is equal to or greater than the reference value, this indicates that the subject will benefit from a regimen for prevention or treatment of kidney disease utilising a binding partner for an anti-PLA2R antibody.

Suitably, the subject may be an individual diagnosed as having kidney disease, where the cause of the kidney disease has not been determined.

Suitably, the method may further comprise implementing a suitable prevention or treatment regimen that has been selected by the method of the invention.

Suitably, the prevention or treatment regimen utilises at least 1 peptide of the invention for use in the prevention or treatment of kidney disease.

Suitably, the method may further comprise obtaining a value representative of the amount of anti-PLA2R antibodies in the subject's body fluid, and comparing this obtained value with a reference value, wherein if the obtained value is larger than the reference value this indicates that the subject will benefit from a regimen for prevention or treatment of kidney disease utilising a peptide of the invention.

In another aspect, the present invention provides a method for monitoring effectiveness in a subject of a treatment regimen for prevention or treatment of kidney disease, the method comprising:
assaying a sample of a body fluid of a subject undergoing a treatment regimen for prevention or treatment of kidney disease, to determine the amount of anti-PLA2R antibodies present in the subject's body fluid sample by a method according to the invention;
comparing the determined amount with a reference value; and
based on this comparison, determining whether the subject's treatment regimen is effective for prevention or treatment of kidney disease.

Suitably, the reference value may be the mean plus three standard deviations of the amount of anti-PLA2R antibody in approximately 30 samples from normal healthy individuals without kidney disease.

Suitably, the reference value may be representative of the amount of anti-PLA2R antibodies present in a comparable sample from the subject at an earlier time point.

Suitably, the subject may be a patient undergoing therapy for kidney disease.

Suitably, in the event that a proposed treatment regimen is identified as ineffective, the regimen may be modified in an attempt to improve effectiveness.

Suitably, in the event that a proposed treatment regimen is identified as ineffective, the regimen may be replaced, and a different regimen tested for efficacy.

Suitably, the subject may be a participant in a clinical trial.

Suitably, the method may further comprise assessment of one or more additional markers indicative of kidney disease.

Suitably, the body fluid may be selected from the group consisting of: blood; serum; plasma; urine; and interstitial fluid.

Suitably, the assay may be a quantitative assay. Suitably, the assay may be selected from the group consisting of: enzyme linked immunosorbent assays (ELISAs); western blotting; fluorescent bead-based immunoassays; and immunofluorescence on cell expressed PLA2R.

Suitably, the assay may be an ELISA that makes use of an immobilised peptide of the invention to capture anti-PLA2R antibodies in a sample, and a labelled antibody directed to the immunoglobulin class to which the anti-PLA2R antibodies belong to detect the captured anti-PLA2R antibodies.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### FIGURES

FIG. 1 shows an assessment of the suitability of mouse 20 monoclonal anti-PLA2R antibody as a surrogate for human autoantibodies. A) Slot blot analysis of NC3 (Ricin like domain, FNII and CTLD 1-3 domains of PLA2R and 28mer (WO 2015/185949) under non-reduced conditions with human and mouse anti-PLA2R Abs. (B&C) immobilised NC3 to GLC chip injected with: B) Human anti-PLA2R autoAbs C) anti-PLA2R mouse 20 Ab concentration of 5-25 nM. Results obtained after reference subtraction and kinetics data fitted to a Langmuir 1:1 interaction model. Association (ka), dissociation (kd), and equilibrium (KD) constants for each run were similar. High binding affinity for NC3 was observed with KD of ˜0.1 nM D) Slot blot analysis of AIP 2, 3, 4, 5, 6, 7, 28mer (SEQ ID NO: 22), head to tail 28mer (SEQ ID NO: 74) and THSD7A peptide (SEQ ID NO: 91) under non-reducing conditions stained positive for both human anti-PLA2R autoAbs and anti-PLA2R mouse 20 Ab. Table depicts the assessment of mouse 20 monoclonal anti-PLA2R Ab and human anti-PLA2R autoAb kinetics

FIG. 2 shows peptide sequence optimization. Phase one peptide sequence optimization was out-sourced to PEPpepPRINT A) Schematic depiction of PEPpepPRINT substitution scan. B) Schematic depiction of the strategy for peptide sequence optimisation. Green indicates amino acids involved in the substitution scan. Highlighted red are peptide platforms 2c and 3c, which successfully bound to the human anti-PLA2R autoAbs C) peptide platform 2c substitutions scan. Substitutions of valine 5 to proline, glutamine 7 to glutamate/aspartate and serine 8 to glutamate/aspartate improves binding. Isoleucine 6 and glutamate 9 critical to autoAb binding D) peptide platform 3c substitutions scan. Most amino acids in the cyclic region can be substituted to improve binding. Substitution of leucine 24 to glutamate/aspartate improves binding the most.

FIG. 3 shows assessment of peptide solubility. Excitation at 280 nm emission at 350 nm measured 0.1 mg/mL 28mer W time course measurement taken at 1, 2, 3, 4, 5 and 24 hours and percentage remaining calculated. 28mer W analysed in water in siliconised, copolymer, homopolymer, borosilicate glass, lobind, nostick polypropylene, standard polypropylene tubes.

FIG. 4 shows the peptide epitope mimic library slot blot. A) Slot blot analysis of peptide epitope library: AIP 2-13, 28mer (SEQ ID NO: 22), 28mer unnatural amino acid linker, 28merW, head to tail 28mer (SEQ ID NO: 74), biotin peptide 2, 20mer (SEQ ID NO: 25), VIQSESLKK (SEQ ID NO: 20), PIQSESLKK (SEQ ID NO: 4), VIDSESLKK (SEQ ID NO: 18), PIDSESLKK (SEQ ID NO: 19), THSD7A peptide (SEQ ID NO: 91), 2a, 2b, 2b mouse, 2c, 2d, 2e, Click chemistry 28mer stained with ponceau S and mouse 20 Ab. B) Slot blot analysis of peptide epitope library Intensity of ponceau S and mouse 20 Ab staining.

FIG. 5 shows the ELISA analysis of peptide epitope library, 1.5 ng of peptide immobilised.

FIG. 6 shows the candidate Peptide Mouse 20 Antibody Binding Kinetics. Immobilised peptide to GLC chip injected with anti-PLA2R Mouse 20 Ab concentration of 5-25 nM. Results obtained after reference subtraction and kinetics data fitted to a Langmuir 1:1 interaction model. Association (ka), dissociation (kd), and equilibrium (KD) constants for each run were similar. High binding affinity for peptides was observed with KD of ˜0.1 nM A) 28mer (SEQ ID NO: 22) B) Head to Tail 28mer (SEQ ID NO: 74) C) THSD7A Peptide (SEQ ID NO: 91) D) Peptide 2 (SEQ ID NO: 35).

FIG. 7 shows the PEPperPRINT Peptides Mouse 20 Antibody Binding Kinetics. Immobilised peptide to GLC chip injected anti-PLA2R Mouse 20 Ab concentration of 5-25 nM. Results obtained after reference subtraction and kinetics data fitted to a Langmuir 1:1 interaction model. Association (ka), dissociation (kd), and equilibrium (KD) constants for each run were similar. High binding affinity for peptides was observed with KD of ˜0.1 nM A: VIQSESLKK (SEQ ID NO: 20) B: PIQSESLKK (SEQ ID NO: 4) C: VIDSESLKK (SEQ ID NO: 18) D: PIDSESLKK (SEQ ID NO: 19) E: 20mer (SEQ ID NO: 25)

FIG. 8 shows phase 2 of the sequence optimisation, specifically the schematic depiction of the strategy for peptide sequence optimisation phase 2. Single underlined amino acids indicate mutations in N-terminal region thought to be beneficial. Doubled underlined amino acids indicate amino acids involved in the substitution scan. Peptide platforms 5, 7, 9e successful bind human anti-PLA2R autoAbs.

FIG. 9 also shows phase 2 of the sequence optimization. In which the top performing peptide substitutions are chosen B) Peptide Platform 5a and e substitution scan. C) PEPPERPRINT substitution scans 7a and e. D) Peptide Platform 9e and 10e substitution scan.

FIG. 10 shows a surface photon resonance inhibition assay (SPR). In which the amount of anti-PLA2R bound to NC3 protein on the chip surface was measured and expressed as a percentage of the control anti-PLA2R solution containing no peptides.

### DETAILED DESCRIPTION

The present invention is predicated on the surprising finding that certain non-naturally occurring peptides have particular utility for use in the treatment of kidney disease via their use as a peptide mimetic or in combination with a device used for apheresis.

In one aspect, the present invention relates to a non-naturally occurring peptide comprising amino acid sequence S-V-L-T-X1-E-N-X2 (SEQ ID NO: 1), wherein X1 is any amino acid and X2 is any amino acid. The present invention has surprisingly found that the sequence SVLTLENC (SEQ ID NO: 21) is a minimal epitope for the autoantibody to PLA2R.

Advantageously, it has been determined that modification at up to positions can increase binding affinity with an autoantibody of PLA2R.

Suitably, X1 may be to D or E. Such peptides may result in improved binding to an autoantibody to PLA2R compared to a naturally occurring fragment of PLA2R.

Suitably, X2 may be C or S.

Suitably, the peptide of the invention may be a variant peptide comprising the amino acid sequence SVLTEENC (SEQ ID NO: 2) or SVLTEENS (SEQ ID NO: 3).

Suitably, the peptide may have enhanced binding affinity to an autoantibody to PLA2R compared to the naturally occurring PLA2R or a fragment thereof. Suitably, and enhance binding affinity may be determined at any appropriate concentration of the autoantibody. Suitably, the binding may be measured as a concentration of autoantibody of less than or equal to 25 nM, or 20 nM or 15 nM or 10 nM or 5 nM. Suitably, the binding of a peptide of the invention to an autoantibody of PLA2R may be compared with the binding of a naturally occurring fragment of PLA2R at an autoantibody concentration of about 5 nM or 10 nM. Suitably, the absolute response may be determined. Suitably, methodology is found in the Examples.

The terms “peptide”, “protein” and “polypeptide” are used interchangeably herein.

The term polypeptide “domain” refers to a portion of a polypeptide sequence that can evolve, function and exist independently of the rest of the polypeptide chain.

Suitably, the peptide may comprise:
A C-terminal domain comprising the amino acid sequence S-V-L-T-X1-E-N-X2 (SEQ ID NO: 1)
An N-terminal domain comprising the amino acid sequence X3-I-X4-X5-E-X6 (SEQ ID NO: 5), wherein X3, X4, X5 and X6 each denote any amino acid.

The present invention has surprisingly determined that the PLA2R has two minimal epitopes important for binding to anti-PLA2R autoantibodies. These are SVLTLENC (SEQ ID NO: 21) and VIQSES (SEQ ID NO: 9). Furthermore, the present invention has surprisingly determined which modifications to these minimal epitopes will provide advantageous properties in a synthetic peptide to be used in apheresis or e.g., as a peptide mimetic.

Suitably, the peptide may be a variant peptide comprising the amino acid sequence S-V-L-T-X1-E-N-X2-K (SEQ ID NO: 6). Suitably, the peptide may be a variant peptide comprising the amino acid sequence S-V-L-T-X1-E-N-C-K (SEQ ID NO: 7). Suitably, X1 may be D or E.

Suitably, the N-terminal domain of the peptide may comprise the amino acid sequence X3-1-X4-X5-E-X6-L-K (SEQ ID NO: 8). Suitably, X3 may be V or P; X4 may be Q, D or E; X5 may be S, D or E and X6 may be S, D or E.

The N-terminus of a protein (also known as the amino-terminus, NH₂-terminus, N-terminal end or amine-terminus) is the start of a protein or polypeptide terminated by an amino acid with a free amine group (—NH₂). By convention, peptide sequences are written N-terminus to C-terminus (from left to right). The C-terminus (also known as the carboxyl-terminus, carboxy-terminus, C-terminal tail, C-terminal end, or COOH-terminus) is the end of an amino acid chain (protein or polypeptide), terminated by a free carboxyl group (—COOH).

As used herein, the terms “N-terminal” and “C-terminal” are used to describe the relative position of e.g. a domain within a polypeptide. Accordingly, a domain that is “N-terminal” is positioned closer (in relative terms) to the N-terminus than to the C-terminus of the polypeptide. Conversely, a domain that is “C-terminal” is positioned (in relative terms) closer to the C-terminus than to the N-terminus of the polypeptide. As used herein, the term “positioned” refers to the location of the e.g. domain within the linear amino acid sequence of the polypeptide.

The terms “N-terminal” and “C-terminal” can be used to describe the relative position of two or more domains within a polypeptide. In this context, a domain that is “N-terminal” is positioned closer (in relative terms) to the N-terminus of the polypeptide than a domain that is “C-terminal”. Conversely, a domain that is “C-terminal” is positioned closer (in relative terms) to the C-terminus of the polypeptide than a domain that is “N-terminal”.

A domain that is “N-terminal” may be, but does not have to be, at the N-terminus of the polypeptide (i.e. it may be, but does not have to be, at the start of the polypeptide terminated by an amino acid with a free amine group). In other words, the first amino acid of an N-terminal domain does not need to be (but may be) the first amino acid of the polypeptide.

This means that there may be other amino acids, polypeptide domains (e.g. tags such as HA tags) etc. between the N-terminus of the polypeptide and the start of the “N-terminal” domain (provided that the domain is positioned closer to the N-terminus than to the C-terminus of the polypeptide; or when used to describe the relative positions of two or more domains, provided that the domain is positioned closer to the N-terminus than a domain that is “C-terminal”).

Likewise, a domain that is “C-terminal” may be, but does not have to be, at the C-terminus of the polypeptide (i.e. it may be, but does not have to be, at the end of the polypeptide terminated by any amino acid with a free carboxyl group). In other words, the last amino acid of a C-terminal domain does not need to be (but may be) the last amino acid of the polypeptide. This means that there may be other amino acids, polypeptide domains etc. (e.g. tags) between the C-terminus of the polypeptide and the end of the “C-terminal” domain (provided that the domain is positioned closer to the C-terminus than to the N-terminus of the polypeptide; or when used to describe the relative positions of two or more domains, provided that the domain is positioned closer to the C-terminus than a domain that is “N-terminal”).

Suitably, the C-terminal domain comprising the amino acid sequence S-V-L-T-X1-E-N-X2 (SEQ ID NO: 1) may be at the C-terminus of the peptide of invention. In addition, or in the alternative, the N-terminal domain comprising the sequence X3-I-X4-X5-E-X6 (SEQ ID NO: 5) may be at the N-terminus of the peptide of the invention.

Suitably, the peptide may comprise a C-terminal domain in which X1 may be L or E and/or X2 may be C or S.

Suitably, the peptide may comprise an N-terminal domain which comprises a variant of the sequence VIQSES (SEQ ID NO: 9) such that one or more of the following substitutions are made: V to P, Q to D or E, optionally either or both S to D or E. For example, the N-terminal domain may comprise the sequence PIDDES (SEQ ID NO: 10) or PIESES (SEQ ID NO: 11).

Linker

Suitably, the peptide comprises a linker between an N-terminal and a C-terminal domain.

As the amino acids between the two minimal epitopes in PLA2R are not required for the binding of anti-PLA2R autoantibodies, any linker which will enable such anti-PLA2R autoantibodies to bind to S-V-L-T-X1-E-N-X2 (SEQ ID NO: 1) or X3-I-X4-X5-E-X6 (SEQ ID NO: 5) may be used. Preferably, the linker allows anti-PLA2R autoantibodies to be able access both.

Suitably, the linker may be selected from a group consisting of: a peptide linker, a synthetic linker, a combination of peptide and synthetic linker.

Suitably, the linker may separate the N-terminal and C-terminal domains by a distance greater than or equal to a linker consisting of 5 glycine residues. Suitably, the linker may comprise 5 glycine residues or may be a synthetic linker comprising a PEG molecule. Suitably, the synthetic linker may further comprise a lysine residue.

Suitably, the linker may be chosen to increase the solubility of the peptide. This would be particularly advantageous when using the peptide of the invention as a mimetic for PLA2R in the treatment of kidney disease. Appropriate linkers to increase the solubility of peptides are known to a person of ordinary skill in the art.

Suitably, the total number of amino acid residues in the peptide may be less than or equal to 28 amino acid residues; or less than or equal to 19 amino acid residues. Advantageously, shorter peptides may have increased solubility.

Suitably, the peptide may comprise the amino acid sequence X3-I-X4-X5-E-X6-PEG-K-PEG-S-V-L-T-X1-E-N-X2 (SEQ ID NO: 12).

Suitably, the peptide may comprise PIESES-PEG-K-PEG-SVLTEENC (SEQ ID NO: 13); VIQSES-PEG-K-PEG-SVLTLENC (SEQ ID NO: 14); VIQSES-PEG-K-PEG-SVLTEENC (SEQ ID NO: 15); PIDDES-PEG-K-PEG-SVLTLENC (SEQ ID NO: 16); PIDDES-PEG-K-PEG-SVLTEENC (SEQ ID NO: 17).

Suitably, the peptide of the invention may be soluble. Suitably, the peptide of the invention may have increased solubility is physiological fluid compared to a naturally occurring fragment of PLA2R which comprises one or both anti-PLA2R autoantibody epitopes.

Nucleic acid molecules encoding a peptide described herein are also provided.

The nucleic acid molecule encoding the polypeptide may be prepared synthetically by established standard methods, e.g. the phosphoroamidite method described by Beucage S. L. et al (1981) Tetrahedron Letters 22, p 1859-1869, or the method described by Matthes et al (1984) EMBO J. 3, p 801-805. In the phosphoroamidite method, oligonucleotides are synthesised, e.g. in an automatic DNA synthesiser, purified, annealed, ligated and cloned in appropriate vectors.

The nucleotide sequence may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R et al (Science (1988) 239, pp 487-491).

The term “nucleic acid molecule” or “nucleotide sequence” as used herein refers to an oligonucleotide sequence or polynucleotide sequence, and variant, homologues, fragments and derivatives thereof (such as portions thereof). The nucleotide sequence may be of genomic or synthetic or recombinant origin, which may be double-stranded or single-stranded whether representing the sense or antisense strand. The term “nucleotide sequence” in relation to the present invention includes genomic DNA, cDNA, synthetic DNA, and RNA (e.g. mRNA) and analogs of the DNA or RNA generated, e.g., by the use of nucleotide analogs.

The nucleic acid molecule can be single-stranded or double-stranded, but preferably is double-stranded DNA, more preferably cDNA for the coding sequence. In a preferred embodiment, the nucleotide sequence per se encoding a peptide properties as defined herein does not cover the native nucleotide sequence in its natural environment when it is linked to its naturally associated sequence(s) that is/are also in its/their natural environment. For ease of reference, we shall call this preferred embodiment the “non-native nucleotide sequence” or “non-naturally occurring sequence”. In this regard, the term “native nucleotide sequence” or “naturally occurring sequence” means an entire nucleotide sequence that is in its native environment and when operatively linked to an entire promoter with which it is naturally associated, which promoter is also in its native environment. Thus, the polypeptide of the present invention can be expressed by a nucleotide sequence in its native organism but wherein the nucleotide sequence is not under the control of the promoter with which it is naturally associated within that organism.

Peptides of the invention are not “naturally-occurring” or “native”. In this regard, the term “native polypeptide” or “naturally occurring polypeptide” means an entire polypeptide or fragment thereof that is in its native environment and/or when it has been expressed by its native nucleotide sequence.

The non-naturally occurring peptides of the invention may be synthesised, in whole or in part, using chemical methods well known in the art (see Caruthers M H et al (1980) Nuc Acids Res Symp Ser 215-23 and Horn T et al (1980) Nuc Acids Res Symp Ser 225-232).

Kidney Disease

Specific examples of “kidney disease” that may be diagnosed, prevented or treated by the methods, uses or medicaments of the invention may be selected from the group consisting of: primary renal failure; membranous nephropathy, such as idiopathic membranous nephropathy, or de novo membranous nephropathy; or recurrent membranous nephropathy.

The binding of anti-PLA2R autoantibodies to PLA2R in vivo is a key mechanism contributing to the development of, and damage associated with, kidney diseases. The inventors believe that the various aspects and embodiments of the invention described herein are able to beneficially reduce such binding, thereby providing a desirable therapeutic effect. Advantageously, the peptides of the invention may have high binding affinity to the autoantibody (e.g. compared to the binding affinity of the autoantibody to the full PLA2R) or a fragment thereof.

Body Fluid Samples

Certain embodiments of the aspects of the invention utilise samples of a body fluid from a subject to determine the amount of anti-PLA2R antibodies present therein.

In a suitable embodiment, the body fluid is selected from the group consisting of: blood; serum; plasma; urine; saliva; and interstitial fluid.

The sample may be one that is obtained from the subject, for example by taking of a blood or interstitial fluid sample, or provision of a urine sample, and this step of obtaining the sample may optionally constitute part of the method in question.

Suitably, the apheresis methods may utilise whole blood.

A Subject

For the purposes of the present invention, a subject may be an individual identified as having, or potentially as having (for example, as at risk of having) kidney disease. The nature of an appropriate subject will be apparent to a skilled reader depending on the aspect of the invention in question.

Merely by way of example, in the case of a method of diagnosis in accordance with the invention, a suitable subject may be one that is considered to be at risk of kidney disease. This assessment of risk may be based upon pre-disposing factors, such as genetic predisposition to kidney disease, or on the exhibition of symptoms consistent with kidney disease. Alternatively, a suitable subject for a method of diagnosis in accordance with the invention may be an individual exhibiting clinical symptoms of kidney disease, but where the cause or nature of the kidney disease has not been identified.

In a method of preventing or treating kidney disease in accordance with the invention, a suitable subject may be one diagnosed as having kidney disease (for example by a method of the invention) or as exhibiting symptoms consistent with kidney disease. Indeed, the subject may be an individual where this regimen for prevention or treatment has been selected as being suitable by a method of the invention.

In the case of the invention's methods of selecting a suitable regimen for the prevention or treatment of kidney disease, a suitable subject may be an individual diagnosed as having kidney disease, but where the cause of the kidney disease has not been determined. In the event that the method of the invention indicates that anti-PLA2R antibodies are present within the subject, this suggests that these antibodies are contributing to the disease process, and that a regimen utilising a binding partner for an anti-PLA2R antibody in a therapeutic manner is likely to be suitable for prevention or treatment of the kidney disease.

In the case of a method of monitoring effectiveness in a subject of a treatment regimen for prevention or treatment of kidney disease, a suitable subject may be a patient undergoing therapy for kidney disease (for example by a method of prevention or treatment of the invention, or by pharmaceutical compositions or medical uses of the invention). In this case, the method of the invention may be used as part of a process of ongoing care for the subject, with a view to optimising a regimen for prevention or treatment of the disease, or ensuring that a selected regimen remains effective. Alternatively, it will be recognised that methods of monitoring effectiveness in a subject are also suitable for use in the development of novel prevention or treatment regimens, such as by clinical trials.

The present invention encompasses methods of preventing or treating kidney disease practiced in subjects requiring such prevention or treatment. A suitable subject requiring such prevention or treatment may be one diagnosed as having kidney disease, or otherwise exhibiting symptoms consistent with the presence of kidney disease.

Some methods of the invention, utilise a peptide of the invention as a binding partner for anti-PLA2R antibodies in a body fluid sample from a subject. In a suitable embodiment a method of this aspect of the invention may additionally employ one or more further binding partners that bind to and retain anti-PLA2R antibodies. Suitably such further binding partners may include full-length PLA2R, or fragments of PLA2R such as the N-C3 fragment described further in the examples.

In one aspect, the invention provides a method of diagnosing kidney disease in a subject, the method comprising:
determining the amount of anti-PLA2R antibodies in a body fluid sample from a subject in accordance with a method of the present invention; and
comparing the determined amount with a reference value,
   wherein if the determined amount of anti-PLA2R antibodies in the sample is equal to or greater than the reference value, this indicates that the subject has kidney disease.

In another aspect, the invention provides a method of selecting a suitable regimen for the prevention or treatment of kidney disease, the method comprising:
determining the amount of anti-PLA2R antibodies in a body fluid sample from a subject in accordance with a method of the present invention; and
comparing the determined amount with a reference value,
   wherein if the determined amount of anti-PLA2R antibodies in the sample is equal to or greater than the reference value, this indicates that the subject will benefit from a regimen for prevention or treatment of kidney disease utilising a binding partner for an anti-PLA2R antibody.

Suitably, the binding partner for anti-PLA2R antibodies may be a peptide in accordance with the present invention.

In a suitable embodiment of a method of the seventh aspect of the invention, the invention provides a method of monitoring effectiveness in a subject of a treatment regimen for prevention or treatment of kidney disease, the method comprising:
assaying a sample of a body fluid of a subject undergoing a treatment regimen for prevention or treatment of kidney disease, to determine the amount of anti-PLA2R antibodies present in the subject's body fluid sample by a method of the seventh aspect of the invention;
comparing the determined amount with a reference value; and
based on this comparison, determining whether the subject's treatment regimen is effective for prevention or treatment of kidney disease.

As discussed further herein, methods in accordance with the seventh aspect of the invention may optionally further comprise a step of implementing a suitable regimen for the prevention or treatment of kidney disease. More details of these embodiments are provided elsewhere in the specification.

Alternatively, or additionally, the methods of the invention may further comprise assessment of one or more additional markers indicative of kidney disease. Examples of such additional markers are considered below.

Diagnosis of Kidney Disease

The present invention relates to methods of diagnosing kidney disease. In suitable embodiments, methods in accordance with this aspect of the invention may be carried out in vitro.

As referred to above, methods of diagnosing kidney disease in accordance with the present invention may be useful for the clinical assessment of individuals considered at risk of kidney disease, individuals suffering a disorder that may be kidney disease, or subjects who have already been identified as having kidney disease, but without the underlying cause of the kidney disease having been determined. In these later cases the diagnostic methods of the invention may be useful in determining that the kidney disease is one associated with the presence of anti-PLA2R antibodies.

A method of diagnosing kidney disease in accordance with the invention involves determining the amount of anti-PLA2R antibodies present in the subject's body fluid sample, and comparing this obtained value with a reference value. It may then be determined whether or not anti-PLA2R antibodies are present in the sample at a greater amount than in the reference value. In such cases, an obtained value greater than the reference value may be indicative that the subject has kidney disease. Details of suitable reference values that may be employed in such embodiments are discussed elsewhere in the present disclosure.

Selection of a Suitable Regimen for the Prevention or Treatment of Kidney Disease

One embodiment of the invention comprises a method for the selection of a suitable regimen for the prevention or treatment of kidney disease. The skilled person will appreciate that there are many different forms and causes of kidney disease, as well as many different agents and regimens for the treatment of such disease. Not all treatment regimens are suitable for prevention or alleviation of all forms of kidney disease.

The aspects of the present invention relating to the selection of a suitable treatment or prevention regimen are based upon the inventor's finding that peptides of the invention can be used to bind to anti-PLA2R antibodies generated by a patient's body against the naturally occurring target protein, and allow the levels of these antibodies in a sample to be determined.

By determining whether anti-PLA2R antibodies are present in a subject these methods of the invention make it possible to identify whether or not the subject in question is one who will benefit from treatment aimed at neutralising or removing these antibodies (and thereby preventing further damage that the antibodies may cause) by use of binding partners to the anti-PLA2R antibodies.

Various optional modifications of the methods of the invention are considered within the present disclosure.

Monitoring Effectiveness of a Treatment Regimen

In one aspect, the invention provides a method of monitoring effectiveness in a subject of a treatment regimen for prevention or treatment of kidney disease. As set out elsewhere, this may involve determining the amount of anti-PLA2R antibodies in a sample from a subject, and comparing this with a reference value. Generally, if the determined value is lower than the reference value this indicates that the treatment regimen is effective. In such cases the lower the determined value, as compared to the reference value, the more effective the treatment regimen.

Methods of the invention in accordance with this aspect are suitable for use in a number of different contexts in which it is wished to assess whether or not a treatment regimen is able to effectively prevent or treat kidney disease.

Merely by way of example, methods in accordance with this aspect of the invention are suitable for use in the field of personalised medicine. Here such methods of the invention are able to determine on a patient by patient basis whether or not a proposed treatment regimen proves effective. In the event that a proposed treatment regimen is identified as ineffective it can be modified in an attempt to improve effectiveness, or replaced, and a different regimen tested for efficacy.

These methods of the invention also allow effectiveness of different methods to be determined and compared with one another, wherein the treatment regimen giving rise to the lowest antibody levels will generally be considered the most effective. Thus, even in the event that a given treatment regimen is determined to be effective by the monitoring methods of the present invention, other treatment regimens that are more effective may be identified, and a decision made to adopt a more effective treatment regimen for future prevention or treatment.

As referred to elsewhere, it will also be recognised that methods of monitoring effectiveness of a treatment regimen in accordance with this aspect of the invention are also suitable for use in the development of novel prevention or treatment regimens. Here a putative treatment regimen may be adopted, and its effectiveness in reducing anti-PLA2R antibody levels assessed, with those that bring about a reduction selected for future use, or for further refinement. Such methods may be useful in the context of clinical trials or the like.

Assaying for Anti-PLA2R Antibodies

Methods the invention may involve determining levels of anti-PLA2R antibodies in a subject by determining the amount of anti-PLA2R antibodies retained by a peptide of the invention.

Once informed by the present disclosure that the peptides of the invention are able to bind and retain anti-PLA2R antibodies, the skilled person will be able to determine a number of suitable methods by which assays to determine the amounts of antibody present may be practiced. The following provides some examples of such assays, as well as guidance regarding factors that may be considered in determining a suitable assay to be used in practicing the invention.

Generally, assays for anti-PLA2R antibodies suitable for use in the methods of the invention will be quantitative in nature.

Suitable assays by which the presence of anti-PLA2R antibodies may be detected, and optionally quantitated, include those selected from the group consisting of: enzyme linked immunosorbent assays (ELISAs); western blotting; fluorescent bead-based immunoassays; and immunofluorescence.

Merely by way of example, in a suitable embodiment the ELISA may use an immobilised peptide of the invention, as a binding partner for an anti-PLA2R antibody, to capture anti-PLA2R antibodies in a sample. The captured anti-PLA2R antibodies may then be detected by means of a labelled antibody directed to the immunoglobulin class to which the anti-PLA2R antibodies belong. For example, in the case of anti-PLA2R antibodies comprising IgG, the presence of such antibodies may be determined by use of a labelled (for example peroxidase labelled) anti-human-IgG antibody. Further details of a suitable embodiment of an assay of this sort are set out in the Examples section of this specification.

In a suitable embodiment assaying of the body fluid sample for anti-PLA2R antibodies is conducted in vitro.

Reference Value

The methods of monitoring effectiveness in a subject of a treatment regimen for prevention or treatment of kidney disease disclosed herein involve obtaining a value representative of the amount of anti-PLA2R antibodies present in a body fluid sample from a subject, and comparing this obtained value to a reference value.

Furthermore, certain embodiments of methods of the invention optionally involve obtaining a value representative of the amount of anti-PLA2R antibodies present in a body fluid sample, and comparing this value to a reference value.

A number of different values may be used as the reference value in such methods of the invention, and selection of an appropriate reference value will determine the nature of the conclusion that may be drawn using the method of the invention.

In certain embodiments of the invention, a suitable reference value may be one representative of a “background” amount of anti-PLA2R antibodies that may be detectable in a sample without indicating the presence of kidney disease. This baseline may reflect the limit of detection of the assay, or the incidence of false positive results. Reference values of this sort may be suitable for use in the methods of the first or second aspects of the invention.

A suitable reference value reflecting a background level of anti-PLA2R antibodies may be established by assaying approximately 30 samples (for example, 25 or more, 26 or more, 27 or more, 28 or more, 29 or more, or 30 or more samples), from normal healthy individuals without kidney disease, to obtain values representative of the amounts of anti-PLA2R antibodies in these samples. In such an embodiment, the reference value (functioning as a threshold between samples considered positive or negative for kidney disease) may be set at the mean value from the healthy individuals, plus three standard deviations (SDs). In such cases an obtained value above the reference value will be indicative of kidney disease in the subject from whom the value has been obtained, while an obtained value below the reference value will indicate that kidney disease is not present in the subject.

The normal healthy individuals may be matched controls with reference to the subject in respect of whom the method of the invention is to be practiced (for example, the healthy individuals may be matched with respect to gender and/or age), or the healthy individuals may be a panel of individuals (for example, a panel of male and/or female individuals having a range of ages that approximately matches the group in whom kidney disease is to be investigated).

In a suitable embodiment of the methods of the invention, the reference value is representative of the amount of anti-PLA2R antibodies present in a comparable sample from the subject at an earlier time point.

In methods of the third aspect of the invention, comparison of the value obtained from the subject with the reference value will provide valuable information as to the effectiveness or ineffectiveness of a treatment regimen. Generally, if the value obtained in respect of the amount of anti-PLA2R antibodies present in the subject's body fluid is lower than the reference value, then this will indicate that the treatment regimen that the subject is undergoing is effective. If the obtained value is higher than the reference value, indicating that the amount of anti-PLA2R antibodies present in the subject's body fluid is elevated as compared to the reference value, this will indicate that the treatment regimen that the subject is undergoing is ineffective. In such cases the treatment regimen may require revision, and the methods of the invention may then be performed again (once the revision has had time to alter the amount of anti-PLA2R antibodies present in the subject) to assess the effectiveness of the revised regimen.

In another embodiment, a first sample is assayed to obtain a first value representative of the amount of anti-PLA2R antibodies present in the subject's body fluid, and a second sample is assayed to obtain a second value representative of the amount of anti-PLA2R antibodies present in the subject's body fluid at a second, later, timepoint, after treatment with a treatment regimen of interest. The first and second obtained values are compared with a reference value indicative of the background level of anti-PLA2R antibodies. If the second obtained value is closer to the reference value than is the first obtained value, then this indicates that the treatment regimen is effective. If the first obtained value is closer to the reference value, or if the two obtained values are the same, this indicates that the treatment regimen is ineffective.

Suitable samples used in the generation of reference values may be of any body fluid, such as sera, as considered elsewhere in the present specification. Samples used for the generation of reference values may by matched, with reference to type, with the sample from which the obtained value is derived. Samples found to contain anti-PLA2R antibodies may be used to create a standard dilution curve as a reference point for comparison with other samples.

Optional Modifications of Methods of the Invention

The methods of the invention may optionally further comprise selecting a suitable treatment regimen in the event that the subject is diagnosed as having kidney disease.

The methods of the invention may optionally further comprise implementing a suitable treatment regimen in the event that the subject is diagnosed as having kidney disease. The implementation may comprise prescribing the selected treatment regimen. Alternatively, the implementation may comprise providing the selected treatment regimen to the subject.

A suitable treatment regimen in the context of the preceding paragraphs may employ the medical use of binding partners for an anti-PLA2R antibody in accordance with the invention, a method of preventing or treating kidney disease in accordance with the invention, or the use of a device for extracorporeal treatment of a patient's blood in accordance with the invention.

Similarly, methods in accordance with the invention may optionally further comprise implementing a suitable prevention or treatment regimen that has been selected by the method of the invention. As above, the implementation may comprise prescribing the selected treatment regimen or providing the selected treatment regimen to the subject. A suitable treatment regimen may employ the medical use of binding partners for an anti-PLA2R antibody in accordance with the invention, a method of preventing or treating kidney disease in accordance with the invention, or the use of a device for extracorporeal treatment of a patient's blood in accordance with the invention.

The methods of the invention may further comprise assessment of one or more additional markers indicative of kidney disease. Such additional markers may be conventional in the art, or may be new markers. Methods utilising combinations of markers may, for example, increase the number of different forms of kidney disease that may be diagnosed by the methods of the invention. Merely by way of example, such additional markers may include antibodies that react with podocyte proteins. Suitably, such additional markers may include one or more markers selected from the group consisting of: anti-IQCJ antibodies; anti-nephrin antibodies; and anti-podocin antibodies.

Prevention or Treatment of Kidney Disease

The present disclosure, addresses the prevention or treatment of kidney disease in a number of contexts: including selection or monitoring of treatment regimens for the prevention or treatment of kidney disease; methods of preventing or treating kidney disease; and medical uses for the prevention or treatment of kidney disease.

Except for where the context requires otherwise, a reference to “treatment” of kidney disease in the present specification should be taken as directed to any effective intervention which has as its purpose the aim of alleviating a disorder manifesting itself as a clinically discernible disease. Treatment, in this context, may encompass any intervention which leads to symptoms of a kidney disease being reduced, or any intervention that prevents the symptoms of kidney disease from worsening in the manner that may be expected if no treatment is undertaken.

For purposes of understanding the present disclosure, references to “prevention” of kidney disease should be taken as directed to intervention initiated before clinical symptoms manifest themselves. Thus, methods of preventing kidney disease will be those that avoid the development of clinical symptoms. It will be appreciated that the diagnostic methods of the invention may be of particular benefit in allowing detection of kidney disease before any clinical symptoms are manifest, thereby permitting the use of methods of the invention in order to prevent kidney disease developing.

The prevention or treatment of kidney disease in may be practiced using binding partners for anti-PLA2R antibodies, and such binding partners are discussed further below.

Methods of prevention or treatment, medical uses, or devices of the invention may make use of binding partners for anti-PLA2R antibodies (such as peptides according to the invention) as the sole therapeutic agent, or as part of a combination therapy. Suitably such combinations may include immunosuppressive drugs as additional therapeutic agents. Examples of such additional therapeutic agents may be selected from the group consisting of: steroids; cyclophosphamide; cyclosporine; and anti B cell monoclonal antibodies. The binding partners for anti-PLA2R antibodies may be provided preceding, at the same time as, or following treatment with additional therapeutic agents such as immunosuppressive drugs.

Binding Partners for Anti-PLA2R Antibodies

Binding partners for anti-PLA2R antibodies encompass any agents capable of binding to such antibodies. In the context of the prevention or treatment of kidney disease, the role of such binding partners is to bind to, and thus reduce the adverse impact of, anti-PLA2R antibodies. Any suitable binding partner capable of achieving this activity may be used. Suitably, binding partners may neutralise anti-PLA2R antibodies (for example by binding to the antibody in a manner that prevents the antibody binding to further PLA2R in the subject), or may reduce the amount of the anti-PLA2R antibodies present in the subject. Suitably, at least one binding partner may be a peptide of the invention.

Suitably, the binding partners utilised will have a preferential affinity for anti-PLA2R antibodies as opposed to other antibodies that may be present. Indeed, the binding partners may be specific for anti-PLA2R antibodies, which is to say that they exhibit minimal, or preferably substantively no, binding of antibodies other than anti-PLA2R antibodies.

The peptides of the present invention are useful as binding partners for anti-PLA2R antibodies. As set out elsewhere in the specification, the peptides of the invention are able to replicate the major antibody-binding activity of PLA2R. However, the use of the peptides of the invention instead of full-length PLA2R in circumstance where it is wished to bind anti-PLA2R antibodies provides a number of significant advantages. Amongst these are that the peptides of the invention are more readily and cost effectively manufactured than full-length PLA2R, and they are also less immunogenic than the full-length protein due to their smaller size. Furthermore, the non-naturally occurring peptides of the invention may have improved binding affinity for the anti-PLA2R antibodies.

Suitably, combinations of different peptides of the invention may be utilised together in a composition or in a device of the invention. By utilising a plurality of different peptides in accordance with the invention the composition and/or device maybe more efficient at binding anti-PLA2R antibodies in a subject or removing anti-PLA2R antibodies. Furthermore, given that there may be variation of anti-PLA2R antibodies in a patient population, a plurality of different peptides in accordance with the invention may be particularly advantageous.

Medical Uses of the Peptides of the Invention

The invention provides peptides in accordance with the present invention for use in the prevention or treatment of kidney disease as one of its' aspects. A peptide of the invention for use in accordance with this aspect of the invention may be provided to a subject requiring such prevention or treatment in a therapeutically effective amount.

The skilled person will be able to determine a therapeutically effective amount of one or more peptides of the invention by a range of measures conventional to this field of the art. Merely by way of example, test amounts of a binding partner for an anti-PLA2R antibody may be provided to the subject, and the effectiveness of these various treatment regimens established by a method in accordance with an aspect of the invention.

In a suitable embodiment, a peptide of the invention for medical use may be provided in a “free” form, in which the peptide is able to move freely within the circulation, bind to anti-PLA2R antibodies, before the peptide-antibody complex is cleared by the body.

In an alternative embodiment, a peptide of the invention for medical use in accordance with the invention (for example in the methods for prevention or treatment of kidney disease in accordance with the invention) may be provided in a form that is adapted to allow the binding partner, and anti-PLA2R antibodies associated with the binding partner, to be retained.

Suitably, a binding partner adapted in this manner may be associated with a retention moiety, such as a magnetic bead or the like.

Alternatively, the binding partner may be adapted by immobilisation on a substrate. Suitably, such a substrate may be part of an immunosorbent column. Further details of such embodiments are described elsewhere in this disclosure.

Methods of Prevention or Treatment of Kidney Disease

The present invention provides methods of preventing or treating kidney disease in a subject that involve the production of an antibody-depleted volume of blood. The depletion of antibodies from the blood removes agents that cause kidney damage, and the resultant lack of damaging antibodies in the subject's circulation enables the prevention or treatment of kidney disease.

The volume of antibody-depleted blood is, in due course, provided to the subject of the treatment. The blood may be returned immediately to the subject, as considered further below, or may be stored prior to administration to the subject.

There are many different ways in which the step of contacting the volume of the subject's blood with a peptide of the invention may be practiced, and suitable examples of these will be readily apparent to those skilled in the art. For instance, the contacting step may be practiced extracorporeally, suitably using a device for extracorporeal treatment of a patient's blood also in accordance with the invention.

In a suitable embodiment, a batch of blood comprising one or more volumes of blood to be treated is removed from the subject, and the steps of contacting a volume of the blood with the peptide of the invention, and subsequent separation of the peptide of the invention and bound antibodies, completed to yield a batch comprising the volume, or volumes, of antibody-depleted blood. Some, or all, of the batch of blood may then be returned to the patient. Alternatively, some or all, of the batch of blood may be stored before return to the patient.

In an alternative embodiment, the steps of contacting the blood with the peptide of the invention, and subsequent separation, are carried out “in line”. Suitably the peptide of the invention is provided in an arrangement so that the patient's blood may flow over the peptide of the invention, thus allowing it to bind anti-PLA2R antibodies in the blood. Continued flow of blood then causes separation of the blood from the retained antibody-peptide of the invention complex, to yield an antibody-depleted volume of blood. The antibody-depleted volume of blood may then be returned directly to the patient, or retained for future use, as above.

It will generally be that case that the subject from whom the blood has been taken will be the subject requiring the prevention or treatment of kidney disease.

Devices for Extracorporeal Treatment of a Patient's Blood

Devices of the invention may incorporate means for separating a peptide of the invention from blood, and such devices represent a suitable embodiment that may be employed in the methods of treatment of the invention.

Suitable means for separating the peptide of the invention from the blood may comprise immobilising means. Such immobilising means may comprise a substrate to which the peptide of the invention is attached. In such embodiments passage of blood over the substrate to which the peptide of the invention is attached will allow antibodies present in the blood to attach to the peptide of the invention, and the substrate will retain the peptide of the invention and bound antibody in place. Relative movement of the blood and immobilised peptide of the invention serves to separate antibodies bound to the peptide of the invention from the blood.

Alternatively, the immobilising means may comprise a retention moiety, such as a magnetic bead or the like, coupled to the peptide of the invention. In this case, the retention moiety may allow immobilisation of the peptide of the invention, and thus separation of the bound antibodies from the blood.

Suitable examples of substrates to which a peptide of the invention may be attached, and thus immobilised, in devices of the invention include those used in immunosorbent columns. Merely by way of example, suitable substrates to which a peptide of the invention may be attached include those selected from the group consisting of: cellulose; cellulose derivatives; agarose; agarose derivatives; polysulphone; polysulphone derivatives; polyacrylamide; polyacrylamide derivatives; and nylon. Such substrates may optionally be provided in forms including hollow fibre cassettes, membranes, or beads. In view of the above, it will be appreciated that immunosorbent columns comprising suitable substrates, such as those constituents or forms listed above, represent preferred embodiments of the devices of the sixth aspect of the invention.

The methods, medical uses, and devices of the invention are suitable for use in a range of subjects. Kidney disease afflicts not only human subjects, but also animals including domestic cats and dogs. Except for where the context requires otherwise, a suitable subject may generally be selected from the group consisting of: a human subject, a feline subject, and a canine subject.

Pharmaceutical Composition

The “term pharmaceutically acceptable carrier” as used herein refers to any suitable diluent, excipient, or a combination thereof, suitable for administration into a subject. A pharmaceutically acceptable carrier may be an organic or inorganic substance, which facilities the delivery of a peptide of the invention to the subject.

In a suitable embodiment, a pharmaceutical composition of the invention may further comprise a pharmaceutically acceptable concentration of salt, buffering agents, and compatible carriers. The compositions may also include antioxidants and/or preservatives. Suitable antioxidants may be selected from the group consisting of: mentioned thiol derivatives (e.g. thioglycerol, cysteine, acetylcysteine, cystine, dithioerythreitol, dithiothreitol, glutathione), tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride.

The pharmaceutical composition of the present invention may be for administration to the subject via any suitable route. A suitable route of administration may be selected from the group consisting of: intravenous injection or infusion. Other methods for administering pharmaceutical compositions will be known to the skilled in the art.

The invention will now be further described, with reference to the accompanying examples and drawings, in which:

### EXAMPLES

Materials and Methods:

Structure and Sequence Mapping

28mer peptide sequences within PLA2R proteins in different animal species (cattle, human, mouse, orangutan and rabbit) and MRF proteins (Endo 180, DEC 205, MR and PLA2R) were analysed using web-based sequence logo generation tool (Seq2Logo) set for p-weighted Kullback-Leibler logo type, heuristics clustering methodology and 50 pseudo count weighting.

Human Embryonic Kidney 293 Cell Culture

Human Embryonic Kidney (HEK) 293-EBNA1 cells were transfected with recombinant PLA2R constructs (NC8 or NC3) expression vectors by the Manchester group. Cells were initially grown in T75 Flasks containing DMEM 4 medium (Sigma D5796), supplemented with 10% foetal calf serum (FCS) and 1% L-glutamine (Q, R-8753) and incubated at 37° C., in 5% CO2 until the cells were confluent. The medium was removed and cells washed with phosphate buffer saline (PBS). The cells were then split with 2 mL of trypsin-EDTA (Sigma T3924) and incubated for 2-3 minutes at 37° C. to dislodge confluent cells from the flask. DMEM 4 (10% FCS 1% Q), was then added to terminate the trypsin reaction. The resultant cells were then divided 1:5 into fresh T75 flasks and incubated at 37° C., in 5% CO2 until the cells were confluent. This procedure was repeated, except 100 mL DMEM 4, 10% FCS 1% Q, was added to terminate the trypsin reaction. Resuspended cells were then transferred into individual three layered cell culture flasks (525 cm2) and incubated at 37° C., in 5% CO2 until the cells were confluent. Media was then removed from each flask and cells washed in PBS before adding 100 mL of DMEM 4 supplemented with 1% Q (Expression medium). Cells were then incubated at 37° C., in 5% CO2, for 3-5 days after which the media was collected in 50 mL tubes, centrifuged at 1400 rpm for 4 minutes, and the supernatant pooled together to 500 mL.

NC3 and NC8 Purification

500 mL expression media containing NC8 or NC3 protein were loaded onto 5 mL Nickel Column (GE Healthcare) and washed with 10 mM Bis-Tris 10 M NaCl 10 mM Imidazole pH 7.2 buffer (Buffer A). Bound proteins were then eluted using a linear gradient from 100% Buffer A to 100% Buffer B (10 mM Bis-Tris 10 M NaCl 500 mM Imidazole pH 7.2) over 6 column volumes (CV). Elution samples with an absorbance >10 A.U. at 280 nm were collected, pooled and loaded onto a Desalting column (GE Healthcare). Bound proteins were then eluted in Buffer C (10 mM BisTris 10 mM NaCl pH 7.2) over 2 CV. Again elution samples with an absorbance >10 A.U. at 280 nm were collected, pooled and loaded onto a 1 mL Q column (BIORAD). Bound proteins were then eluted in a gradient buffer from 100% Buffer C to 100% Buffer D over 2 CV and 0.5 mL fractions collected. NC8 and NC3 proteins were eluted at ˜150 mM NaCl. Samples were then analysed by SDS PAGE for purity and concentration measured by 280 nm spectrophotometer using the extinction coefficient (0.1%) 2.40 or 1.88 respectively.

Human Anti-PLA2R AutoAb Purification

A PD10 Desalting column (GE Healthcare) was calibrated with 6 CV of milli Q water. 2.5 mL of NC3 protein in 10 mM Bis-Tris 150 mM NaCl pH 7.2 was added to the column and was eluted in 3.5 mL of NHS HP HiTrap column coupling buffer (0.2 M NaHCO3 0.5 M NaCl pH 8.3). 1 mg of NC3 protein, in NHS HP HiTrap column coupling buffer, was cross-linked to a 1 mL NHS HP HiTrap column (GE Healthcare). NHS HP HiTrap column was washed with 6 CV 1 mM HCl, then 1 mL of 1 mg NC3 was immediately added and incubated for 30 minutes at room temperature. 6 CV of deactivating buffer (0.5 M ethanolamine, 0.5 M NaCl pH 8.3) followed by 6 CV of acetate buffer (0.1 M acetate, 0.5 M NaCl pH 4) and a further 6 CV deactivating buffer were injected sequentially and incubate for 30 minutes at room temperature. The column was washed with 6 CV acetate buffer, 6 CV deactivating buffer A, 6 CV acetate buffer and 6 CV of PBS containing azide. IMN patient Serums (Nottingham 28, Oxford 10, Dundee 01) were diluted 1:2 with PBS and loaded overnight onto a 1 mg NC3 NHS HP HiTrap column. AutoAbs were eluted in PBS containing 100 mM glycine and 0.5 mL fractions were collected in tubes containing 50 uL 1 M Tris-HCL. AutoAb samples were analysed by SDS-PAGE, pure samples selected, pooled and concentration measured by 280 nm spectrophotometry using extinction coefficient (0.1%) 1.36.

Generation of Anti-PLA2R Mouse 20 Ab

Anti-PLA2R mouse 20 Ab was generated by ProteoGenix through standard hybridomas techniques. Mice were immunised to NC3 and then immune cells merged with sp12 myeloma cells. Positive anti-PLA2R secreting hybridomas were selected and antibodies screened for reactivity with NC3.

SDS PAGE and Western Blot Analysis

Samples were solubilised in sample buffer (Sigma), containing 100 mM Dithiothreitol (DTT) if samples were reduced, heat denatured at 95° C. for 5 minutes before loading onto 4-12% Bis/Tris SDS PAGE gel (Invitrogen Life Technologies) in a XCell II™ blot module western blot kit (Invitrogen Life Technologies). NuPAGE MES running buffer (Invitrogen Life Technologies) was added to the gels and a current of 180 V applied for ˜45 minutes. Proteins were then either visualized by Coomassie staining or processed for western blot analysis.

Western Blot and Slot Blot Analysis

Nitrocellulose membranes (Whatman International) for western blot analysis were prepared by transferring SDS PAGE gel proteins to a 0.45 nm nitrocellulose membrane and a current of 30 V applied for 1 hour in 1× transfer buffer (25 μM Tris-Hcl, 20% methanol, 0.01% SDS, 190 μM glycerol). For slot blot analysis, 1 μg of recombinant PLA2R protein or 10 μg of peptide was loaded onto slot blot wells and vacuumed onto a 0.45 nm nitrocellulose membrane prewashed in mili Q water. Where the protein or peptide was to be reduced 100 nM DTT was added to the sample before loading onto the well. Nitrocellulose membranes from both techniques were than stained by ponceau S solution (0.1% [w/v] ponceau S, 5% [v/v] acetic acid) and images captured. Membranes were then washed in PBS with 0.05% Tween (PBST) and blocked with 1× blocking buffer (Sigma B6429) for 1 hour then washed three times in PBST. Primary Abs (human anti-PLA2R AutoAbs 1:100 or mouse 20 anti-PLA2R Abs 1:1000) in PBST were then incubated for 1 hour and washed three times in PBST. Secondary Abs (Goat anti-mouse 680—Alexa Fluor; and mouse anti-human 680—Alexa Fluor supplied by ProteoGenix) in PBST were incubated for 1 hour in the dark before washing three times in PBST followed by a mili Q water wash. All incubations involved gentle agitation at room temperature. Membranes were then scanned using LI-COR Odyssey Imaging System. Images were then analysed by ImageJ Fiji (Schindelin et al., 2012). Peptides were supplied by Proimmune Ltd., ProtoGenix, Cambridge Research Biochemicals (CRB) and Peptide Synthetics.

SPR (Surface Plasmon Resonance) Analysis

NC3 protein and peptides (20 μg/mL) in 10 mM sodium acetate pH 4.5 were immobilised onto individual ProteOprin GLC sensor chip lanes using standard EDCNHS amine coupling and 1 M ethanolamine blocking. Concentrations of 25, 20, 15, 10 and 5 nM of either mouse 20 or human anti-PLA2R Abs in HEPES buffer (10 mM HEPES 150 mM NaCl 0.05% Tween pH 7.2) were injected for 120 seconds at a flow rate of 70 μL/min with an 800 second dissociation phase. Two injections (40 seconds) of 10 mM NaOH regenerated GLC chip back to baseline levels. Samples were double referenced with a blank row containing only HEPES buffer and a blank lane. The results were analysed and fitted to a Langmuir 1:1 model.

Peptide Sequence Optimisation

PEPperPRINT manufactured two PEPperCHIP® Peptide Microarrays corresponding to two distinct phase of peptide sequence optimisation (described in the results). The microarrays were pre-stained with the secondary Ab goat anti-human IgG conjugated to DyLight680 (1:5000) and control Ab anti-HA conjugated to DyLigh800 (1:2000) in incubation buffer. Peptide sequence optimisation phase one PEPperCHIP® Peptide Microarray was incubated with human plasma, from a Nottingham patient, at dilutions of 1:100 in incubation buffer. In phase two the PEPperCHIP® Peptide Microarray was incubated with human plasma from Nottingham, Oxford and Dundee patients at dilutions of 1:100 in incubation buffer. After washing, the microarrays were stained with the secondary antibody, used in the pre-staining, followed by data read-out with a LI-COR Odyssey Imaging System.

Peptide Concentration Analyses

The concentration of peptides (AIP 9, 10 and 11, three repeats) which had been reconstituted in PBS was determined by the following approaches. Absorbance at 280 nm was measured by spectrophotometer and NanoDrop using an extinction coefficient (0.1%) of 1.746, 1.960 or 2.194 respectively. Absorbance at 205 nm was measured by NanoDrop and converted to mg/mL using the extinction coefficient (0.1%) 31. Bicinchoninic acid assay (BCA) was used to evaluate stock concentrations of BSA (Bovine Serum Albumen) of 2000, 1500. 1000, 750, 500, 250, 125 and 25 μg/mL in PBS. 25 μL of each of the stock solutions and peptide samples were added to 200 μL Pierce® working reagent (Pierce® BCA Protein Assay Reagent A 50:1 Reagent B) and incubated at 37° C. whilst shaking for 30 minutes. Absorbance was measured at 570 nm using a well plate reader. A calibration curve was developed from stock solution readings and peptide concentrations interpolated. Direct Detect® analysis of 2 μL peptide samples placed onto a micro detect card was used to determine concentration using a pre-set calibration curve.

Optim Analysis

31mer peptide solutions (0.3 mg/mL) reconstituted in mili Q water were placed in a micro-cuvette array and inserted into an Avacta Optim 1000. The samples were excited at 280 nm and emissions at 350 nm measured over a temperature range of 10-90° C.

Fluorometer Analysis

31mer peptide solubility was determined by diluting to 0.1 mg/mL in three buffers (NaPO4; HEPES pH 8.5 and water) and incubated at 4° C. over 16 hours. Samples were withdrawn and diluted 1:6 in mili Q water at the beginning and end of the incubation period. The samples were excited at 280 nm and emissions at 350 nm measured by a Fluoro Max-4 fluorometer. 28merW peptide solubility was determined by diluting to 0.1 mg/mL in two buffers (water and PBS) into seven different tubes (siliconised, Fisherband; copolymer, Axygen; homopolymer, Axygen; borosilicate glass, National Scientific; LoBind, Sigma; NoStick polypropylene, Apex, standard polypropylene tubes, Sigma) and incubated at 4° C. over 24 hours. Samples were withdrawn, diluted (1:6 mili Q water) and evaluated following 1, 2, 3, 4, 5 and 24 hour incubation periods. The samples were excited at 280 nm and emissions at 350 nm measured by a Fluoro Max-4 fluorometer.

ELISA (Enzyme-Linked Immunosorbent Assay) Analysis

ELISA 96 well plates (ThermoScientific, 3455) were coated with 100 μL of 1.5 μg/mL peptide in 0.1M NaHCO3 pH 9.5 buffer overnight at 4° C. The solution was removed and wells blocked with 100 μL of superblock (ThermoScientific, 37515) for 1 hour. The solution was again removed and replaced with 100 μL of 2 μg/mL of mouse 20 Ab in superblock containing 0.05% Tween. The wells were then incubated for 2 hours at room temperature with constant shaking. The solutions were removed and washed nine times in PBST and then replaced with 100 μL horseradish peroxidase (HRP) conjugated goat anti-mouse IgG 1:5000 in superblock containing 0.05% Tween. The wells were again incubated for two hours at room temperature with constant shaking. The solutions were removed and washed nine times in PBST. 100 μL tetramethylebenzadine (Sigma, T4444) was added and incubated for 10 minutes at room temperature. 50 μL of sulphuric acid and absorbance measured on a well plate reader at 450 nm. Controls of peptide with secondary Ab and primary and secondary Ab were prepared.

MST (Microscale Thermophoresis)

Mouse 20 anti-PLA2R Ab was buffer exchanged from a 100 mM glycine, 10 mM Tris buffer to PBS using the spin column A buffer exchange protocol. 3 mL of mili Q water was added to spin column A which was then agitated and centrifuged at 1500×g for 1 minute. 300 μL of PBS was added to spin column A which was agitated and centrifuged at 1500×g for 1 minute. This step was repeated three times. 100 μL of mouse 20 anti-PLA2R Ab was injected onto the column and eluted by centrifugation (1500×g for 2 minutes) in PBS. 100 μL of 10.5 μM mouse 20 anti-PLA2R Ab was mixed 1:1 with 30 μM fluorescent dye solution and incubated in the dark for 30 minutes. Column B was equilibrated in MST buffer (150 mM NaCl, 50 mM Tris; 10 mM MgCl2; 0.05% Tween) and mouse 20 Ab conjugated with a fluorescent dye at 2:3 in MST buffer added. The fluorescent dye linked Ab was eluted in 600 μl MST buffer. 1:50 dilution of mouse 20 Ab conjugated with a fluorescent dye was incubated with either 28mer peptide or NC3 protein in 16 1:2 serial dilutions from 1000 nM-3 μM. The normalised fluorescence (Fnorm) was measured and plotted against concentration of 28mer peptide or NC3 protein to produce a binding curve. Kinetics were then determined using a Compared Kd-fit analysis. All columns and fluorescent dyes were supplied by NanoTemper Technologies.

Q-Sense

A silicone dioxide chip was loaded into the Q-Sense Explorer and exposed to a Q-Sense buffer (10 mM HEPES, 150 mM NaCL pH 7.4) at a flow rate of 70 μL/min. 20 μM of the 28mer in Q-Sense buffer was injected and immobilised onto the chip. 50 nM Mouse 20 anti-PLA2R Ab or a rabbit anti-PLA2R Ab control were injected onto the chip and then regenerated with 10 mM NaOH. Measurements of the change in dissipation and frequency involved in the binding interaction between the 28mer peptide and the Abs were recorded.

Results:

Purification and Validation of Autoantibodies to the Major Epitope of Human PLA2R: Recombinant PLA2R Purification

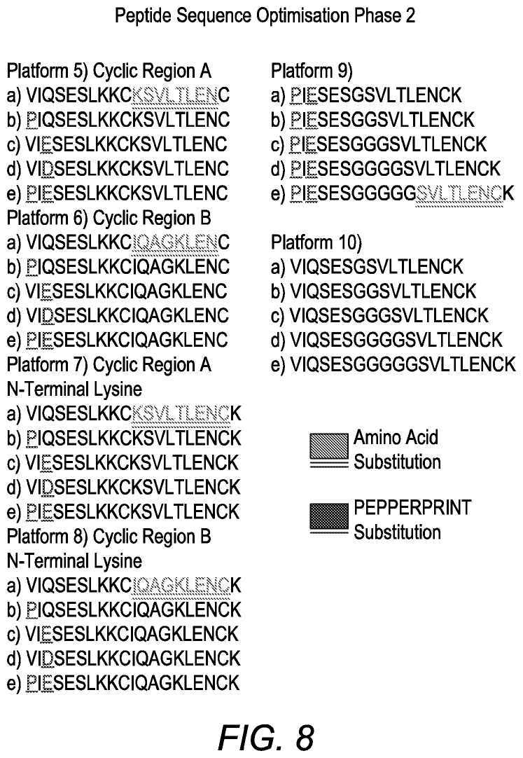

Recombinant PLA2R proteins, NC8 and NC3, were expressed in HEK 293 cells and purified by a three-step column method. NC8 contains a Ricin, Fibronectin type 2 and eight CTLD domains, whereas, NC3 contains a Ricin, Fibronectin type 2 and only three CTLD domains (FIG. 8 A). Approximately 2 mg NC8 and 8 mg NC3 were purified from 1 L of collected expression media. Both recombinant proteins showed similar levels of purity by non-reducing SDS-PAGE gel analysis (data not shown) and their positions on the gel corresponded to the molecular weight of each construct (108 kDa NC8 and 90 kDa NC3). Recombinant PLA2R proteins were acquired for use in the purification of human anti-PLA2R autoAbs.

Purification of Human Anti-PLA2R Autoantibodies

Human anti-PLA2R autoAbs were purified by immobilising purified NC3 HiTrap NHS-activated HP columns. Three IMN patient sera (Oxford, Nottingham and Dundee) were affinity purified via individual 1 mg NC3 immobilised columns. 300 μg/ml, 210 μg/ml and 170 μg/mL of autoAbs were affinity purified from the Nottingham, Oxford and Dundee patient sera respectively. Non-reducing SDS-PAGE analysis of purified Anti-PLA2R autoAbs produced two bands. One low intensity band at 150 kDa, which was thought to be the fully formed antibody and a secondhigh intensity band at 75 kDa, assumed to be half the autoAb one heavy and light chain (Aucan et al., 2000, Davies et al., 2014). IMN patient autoAbs belong predominantly to the IgG4 subtype, which are known to have a predisposition for swapping a heavy chain, known as Fab arm exchange, resulting in bispecific or monovalent Abs (Aucan et al., 2000, Davies et al., 2014). Alternatively, it is possible that the autoAbs are destabilised via the purification process.

Validation of Human Anti-PLA2R Autoantibodies Binding to the PLA2R Major Epitope Region

To validate if purified autoAbs recognise the major epitope, western blots and slot blots were performed under reducing and non-reducing conditions. Western blot evaluation indicated that the human autoAbs only detected NC8 and NC3 when the samples were non-reduced (data not shown). Slot blot results indicated that both NC8 and NC3 were detectable under reducing and non-reducing conditions but the major epitope peptide mimic (28mer) was not detectable under reducing conditions. The loss of reactivity under reducing conditions of NC8, NC3 and the 28mer indicates that the autoAbs recognised a conformational epitope within all three peptides.

Assessment of the Suitability of Mouse 20 Monoclonal Antibody Binding PLA2R Constructs as a Surrogate for Human Autoantibodies

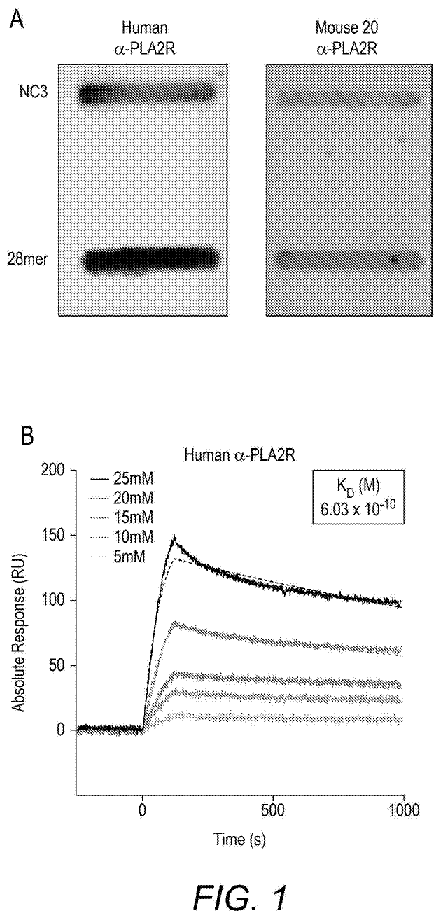

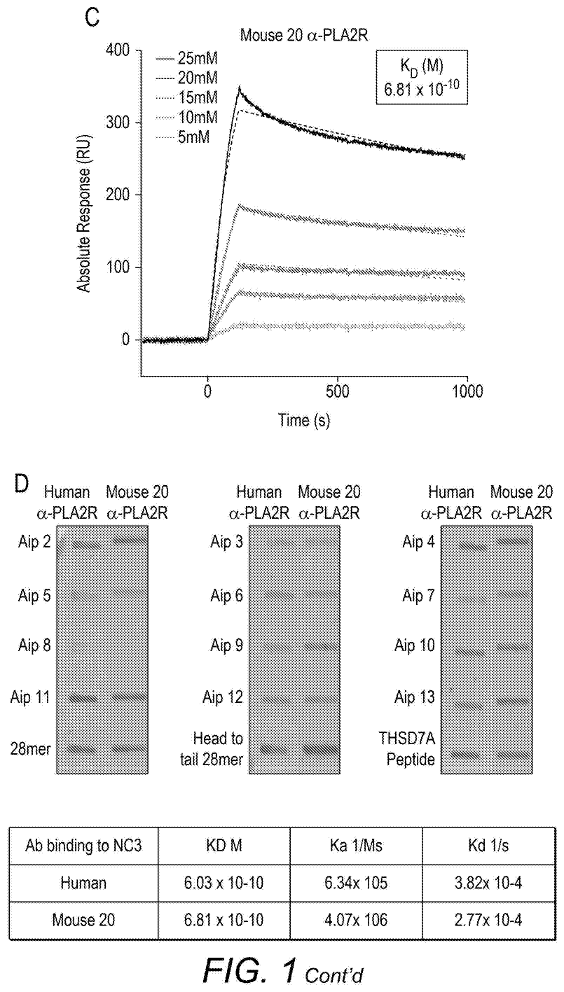

A mouse monoclonal anti-PLA2R Ab, mouse 20, was investigated as a potential substitute for the human anti-PLa2R autoAbs to overcome previously described issues of low concentration, volume and potential degradation. Slot blot evaluation of mouse 20 and human anti-PLA2R autoAbs interaction with NC3 and the 28mer revealed comparable binding (FIG. 1 A), suggesting that the mouse 20 and human anti-PLA2R Abs are binding similar regions of PLA2R. However, these results are qualitative only with no information on relative binding kinetics.

Surface plasma resonance (SPR) was undertaken to ascertain the relative binding affinities of mouse 20 and human Abs (FIGS. 1 B and C). Both mouse and human anti-PLA2R Abs were immobilised, by EDC/NHS activation, onto a separate lane of a ProteOn GLC chip. NC3 was injected onto the chip at concentrations of 25, 20, 15, 10 and 5 nM. The SPR investigation showed similar binding affinities between human autoAbs and mouse 20 Abs to NC3 with the equilibrium constants (KD) of 0.603 and 0.681 nM respectively. The association (Ka) and dissociation constants (Kd) had similar values shown in Table 1. The binding of both antibodies appeared to have a strong affinity for the NC3 having a low off rate up to 20 nM. At high concentrations (25 nm), however, NC3 appeared to show a 2-state off rate with an initial fast dissociation rate followed by a more predicted low dissociation rate. This observation could be a consequence of NC3 self-association leading to a high initial dissociation rate, which is known to occur at higher concentrations of NC3, leading to potential inaccuracies in binding kinetics measurements. These results indicate equivalent binding affinity towards NC3 between human and mouse antibodies.

Mouse 20 Ab and human autoAbs binding characteristics across a range of major epitope peptide mimics (AIP 1-7, 28mer (SEQ ID NO: 22), head to tail cyclic 28mer (SEQ ID NO: 74) and THSD7A (SEQ ID NO: 91); data not shown) were assessed via slot blot. This analysis was designed to determine whether subtle differences in peptide sequences would illicit similar interactions with either Ab (FIG. 1 D). Overall the mouse 20 Ab was found to have a similar binding profile to the human autoAbs. Subtle differences were observed for AIP 7, which had a slightly higher Ab staining intensity with the mouse 20 Ab (Results FIG. 1 D).

Peptide Sequence Optimisation—Phase 1

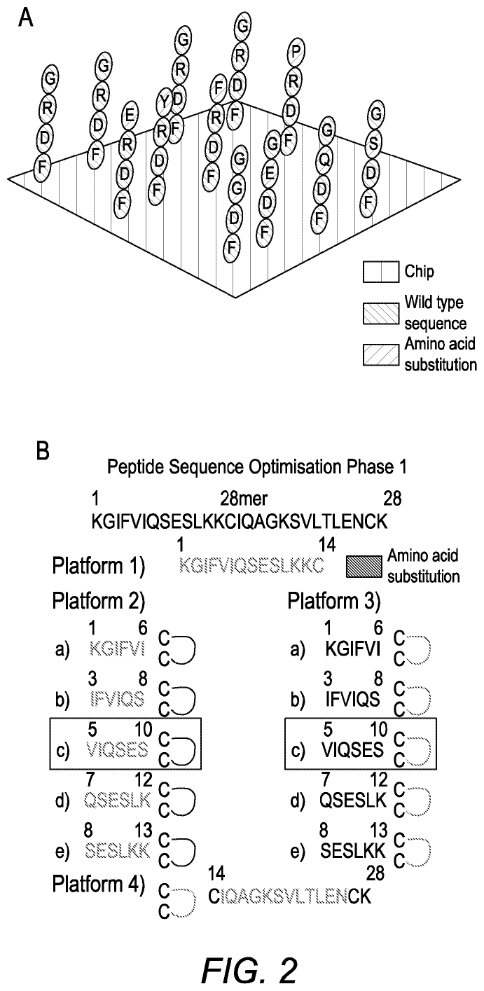

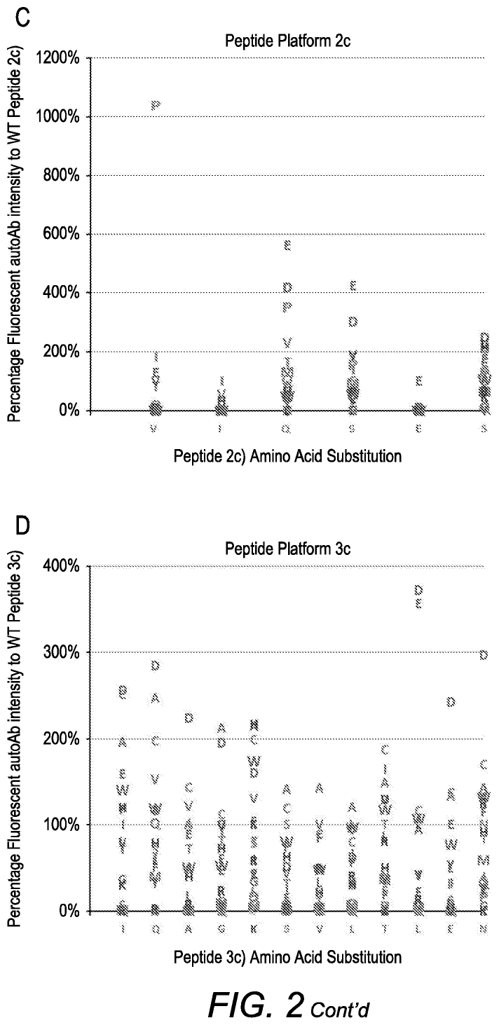

Work to identify the critical amino acids within the 28mer sequence (SEQ ID NO: 22) was out-sourced to a peptide microarray company, PEPperPRINT®. They performed epitope substitution scans (PEPperPRINT, 2014b, PEPperPRINT, 2014a) in which each amino acid was substituted with all 19 other amino acids in a sequential manner, (FIG. 2 A). The peptide microarray assay was then tested with 1 mL of 300 ug/mL purified human Nottingham patient autoAbs. Several peptide platforms were designed to cover the whole 28mer sequence (KGIFVIQSESLKKCIQAGKSVLTLENCK (SEQ ID NO: 22)). Peptide platform 1 comprised the linear part of the 28mer, amino acids 1 to 14, where all amino acids highlighted in green are substituted (FIG. 2 B). Peptide platform 2 consisted of five linear six amino acid peptides, in a frame shift of two positions, of the 28mer linear sequence (amino acids 1-13 KGIFVIQSESLKK (SEQ ID NO: 23)) added to a full cyclic region of the 28mer (amino acids 14-27 CIQAGKSVLTLENC (SEQ ID NO: 24)). Amino acid substitutions were in the linear part of the 28mer only, highlighted in green in FIG. 2 B. Peptide platform 3 as per platform 2 but with the amino acid substitution in the cyclic region. Finally, peptide platform 4 comprised the cyclic region only with amino acid substitutions from position 15-27, FIG. 2 B. Peptide platforms 2c and 3c (VIQSESCIQAGKSVLTLENC (SEQ ID NO: 25) called 20mer, highlighted red in FIG. 2 B) were shown to successfully interact with the human Nottingham patient autoAb. The peptide platforms were then evaluated in more detail to determine critical amino acids for the epitope and additional beneficial amino acids to improve binding characteristics, FIGS. 2 C and D.

Peptide Platform 2c Substitution Scan

The implications of the substitution of amino acids at positions 5-10 within the linear sequence of peptide platform 2c on fluorescent Ab binding intensity compared to WT peptide C are shown in FIG. 2 C. Exchanging valine at position 5 for proline increased the interaction with the Nottingham patient human autoAb from 100 to >1000%. Substitution of glutamine at position 7 with glutamate or aspartate was shown to increase binding to over 500 and 400% respectively. Similarly, exchanging serine at position 8 for glutamate or aspartate provided a moderate increase in binding to over 400 and 300% respectively. Serine at position 10 does not appear to be important to overall binding as substitution by almost all amino acids provided minimal improvements in binding. However, substituting isoleucine at position 6 and glutamate at position 9 with any other amino acid is deleterious to autoAb binding suggesting they are critical components of the epitope.

Peptide Platform 3c Substitution Scan

FIG. 2 D, illustrates the implications of substituting amino acids at positions 15-26 within the cyclic region of peptide platform 3c. The results indicate that almost every amino acid within this region could be replaced by at least one other amino acid to improve binding performance. However, it does appear that exchanging leucine at position 24 for either aspartate or glutamate generated the most significant increase in binding of >300%.

Consequently, optimised linear sequences, without the cyclic chain, (VIQSESLKK (SEQ ID NO: 20), PIQSESLKK (SEQ ID NO: 4), VIDSESLKK (SEQ ID NO: 18), PIDSESLKK (SEQ ID NO: 19) were selected for further evaluation. The peptides were specifically designed to test the impact of amino acid substitutions identified to be beneficial to binding in peptide platform 2c. Additionally, comparing the 20mer (SEQ ID NO: 25), which contains the cyclic region, to linear peptides enables the clarification of the importance of the cyclic region for autoAb binding.

Overcoming Limitations of Peptides

Preliminary analyses, by slot blot and ELISA to determine levels of interaction with the autoAbs with newly designed peptides (PEPperPRINT peptides) were unsuccessful. Several approaches to optimise the protocol were investigated including varying concentrations of Abs, membrane type and pore size all of which were found to have no effect on the outcome. Two hypothesis were drawn 1) the current direct detect technique to measure peptides concentration was inaccurate, resulting in peptide concentrations below the sensitivity of the assay. 2) Subtle differences in peptide sequences affect the level of association to the nitrocellulose membrane. Additionally, widely reported issues with peptide solubility were found to dramatically reduce the concentration of peptides in solution. Work to improve the measurement of peptide concentrations, assessment of peptide association to nitrocellulose and optimisation of peptide solubility were also explored.

Peptide Concentration

Peptide concentrations are usually determined using a 280 nm UV spectrophotometer, however, this technique requires the presence of aromatic amino acids such as tryptophan. Unfortunately, the majority of peptides within our library do not contain aromatic amino acids and therefore the Direct detect technique, which uses infrared to detect amide bonds present in all peptides and proteins, was thought to be a more appropriate alternative. Two additional peptide concentration techniques (205 nm NanoDrop and BCA assay) were evaluated alongside Direct detect compared to 280 UV spectrophotometer evaluation to identify the most accurate method to determine peptide concentrations for this library. The 205 nm NanoDrop approach measures amide bonds within the UV range and the BCA assay interpolates peptide concentrations from a BSA standard concentration curve. A 280 nm NanoDrop technique was included as a positive control. Tryptophan containing peptides (AIP 9, 10 and 11) were used to assess the accuracy of the four techniques, (data not shown).

As expected the positive control the 280 nm NanoDrop reading gives the most accurate and precise results compared to the 280 nm UV spectrophotometer (data not shown). The BCA Assay and Direct detect methods displayed low accuracy and precision (˜1 mg/mL and ˜0.5 mg/mL deviation respectively) collectively across samples and at an individual peptide level. The results confirm hypothesis 1) that the direct detect technique is not sufficiently accurate to assess peptide concentration. However, the 205 nm NanoDrop technique was shown to deliver high accuracy and precision at an individual peptide level and across all samples with a deviation of ˜0.1 mg/mL and was taken forward in further studies.

Assessment of Peptide/Membrane Association

Using the 205 nm NanoDrop peptide concentration technique peptide association with a nitrocellulose membrane was assessed by ponceau S staining, which is a reversible diazo dye that binds to peptide and protein materials. The band intensity of some of peptides from the library, e.g. PEPperPRINT peptides, was weak to non-existent suggesting poor to no association with the nitrocellulose membrane. Whereas others such as the 28mer generated high band intensity indicating strong association. Consequently, the variability in peptide association to nitrocellulose membranes confirms hypothesis 2) and highlights the need for additional techniques to assess the breadth of the peptide library (data not shown).

Assessment of Peptide Solubility

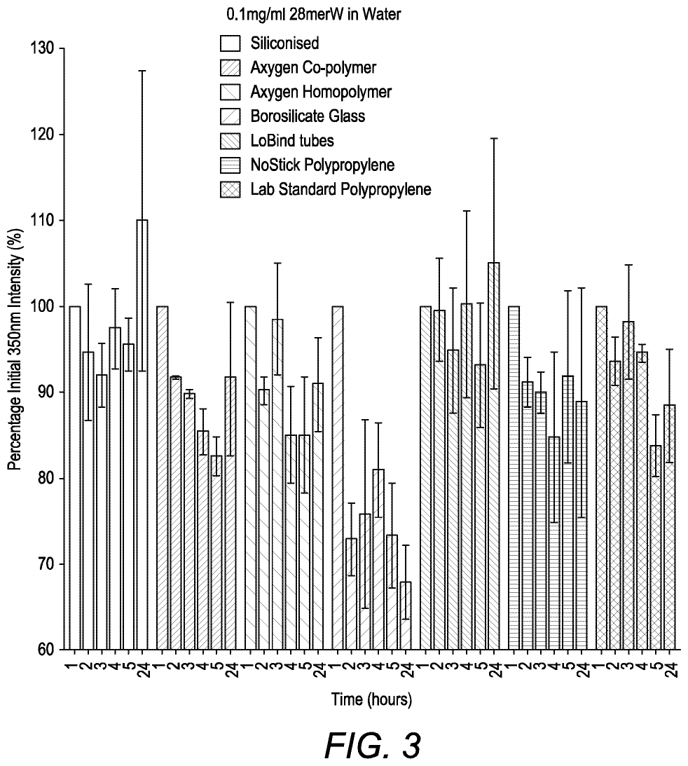

Peptide solubility issues including, formation of aggregates in test solutions and adherence to surfaces, such as test tubes, were found to significantly reduce available concentrations of active peptide in solution. The solubility of a 31mer peptide containing a tryptophan residue, known to bind to the autoAb, was evaluated via Optim (excitation at 280 nm and emission at 350 nm) to determine the correlation of peptide solubility with temperature (data not shown). The solubility of the 31mer in water, showed a reduction in peptide concentration with increasing temperature with only ˜20% of the peptide remaining in solution at 90° C. A sharp loss of peptide in solution (˜20%) was observed between 20-30° C. which was unexpected. Bearing in mind that the Optim test takes ˜30 minutes to achieve a 10° C. increase, the rapid initial loss of ˜20% of the peptide in solution must be both temperature and time dependent. Work to confirm whether time in solution was a major factor and also whether peptide solubility could be improved by introducing a range of buffers (water, sodium phosphate and HEPES pH 8.5) was explored by fluorescence emission at 350 nm, one and 16 hours following reconstitution. Data not shown illustrates that after 16 hours incubation all test solutions counts per second (CPS) intensity at 350 nm was below ˜1% indicating the 31mer is unstable irrespective of the presence of buffers. A 28merW peptide, containing a tryptophan substitution of leucine at position 24, was evaluated to determine whether a peptide closer in sequence to the 28mer has inherently better olubility characteristics. In addition, the solubility of 28merW in seven different test tube materials (siliconised, copolymer, homopolymer, borosilicate glass, LoBind, NoStick polypropylene, standard polypropylene tubes) was evaluated by fluorescence emission at 350 nm over a 24 hour time course (1, 2, 3, 4, 5 and 24 hours) at 4° C. The results (FIG. 3) indicate that the 28mer W was more soluble than the 31mer with 89% and 85% of the peptide remaining in solution after 24 hours in water and PBS respectively. Of the seven different test tube materials evaluated (FIG. 3) peptide solution in siliconised and LoBind tubes were found to maintain ˜100% 350 nm intensity over the 24 hour period. Solutions in copolymer, homopolymer, NoStick polypropylene and standard polypropylene tubes lost <20% peptide over the 24 hour period. Conversely, ˜25% of the peptide was lost within an hour in Borosilicate glass tubes.

Candidate Peptide Assessment

The full peptide library was reassessed using the optimised techniques identified in 4.5 to ensure peptides were not falsely eliminated by the Manchester group analysis.

Peptide Epitope Mimic Library Slot Blot Analysis

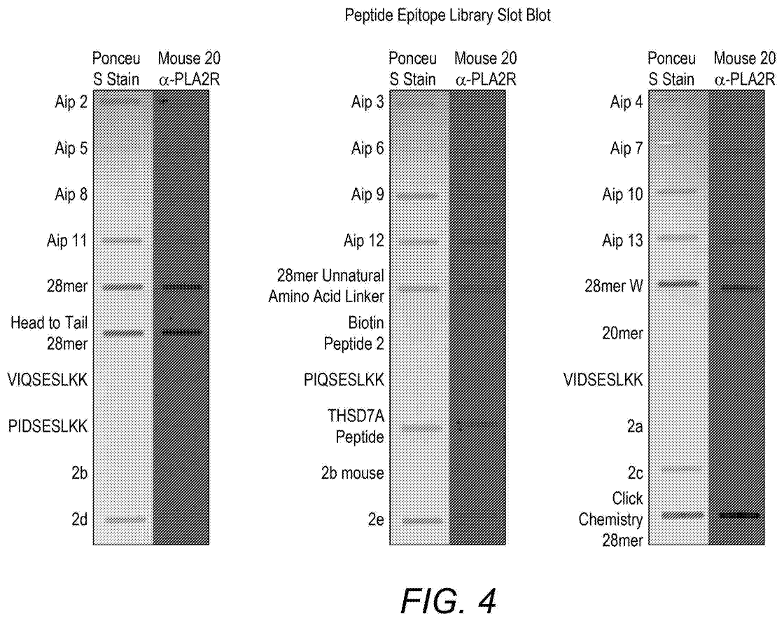

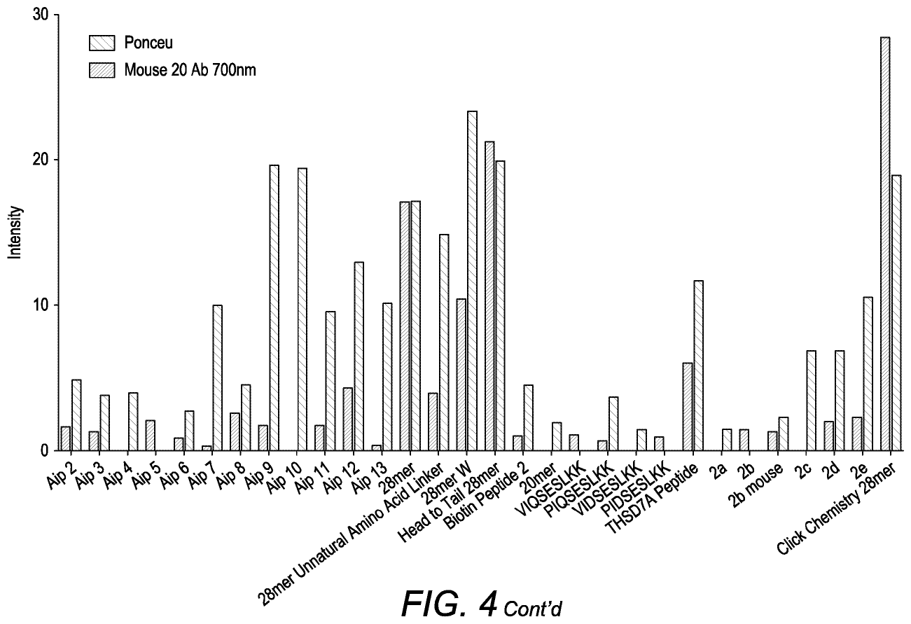

Incorporating the 205 nm NanoDrop and ponceau S staining the assessment of the peptide epitope library was undertaken with the mouse 20 Ab. The results of the slot blot analysis are shown in FIGS. 4 A and B. ˜47% (14/30) of the peptide library did not associate with the nitrocellulose membrane (AIP 4, 5, 6, 7, 8, biotin peptide 2, 2a, 2b, 2b mouse, and PEPperPRINT peptides) and could not be assessed by this technique. This analysis indicates that ˜53% (16/30) of the peptide library stained positive, with the ponceau S, indicating presence on the membrane. Eight of these peptides were not found to bind to the mouse 20 Ab (AIP 9, 10, 11, 12, 13, 2c, 2d, and 2e). Eight ponceau S positive peptides (AIP 2, 3, 28mer, unnatural amino acid linker 28mer, 28mer with a tryptophan (28mer W), head to tail 28mer (SEQ ID NO: 74), THSD7A peptide (SEQ ID NO: 91) and click chemistry 28mer) were found to interact with the mouse 20 Ab. The 28mer, 28mer W, head to tail and click chemistry 28mers interacted with the highest intensity of all peptides evaluated.

Peptide Epitope Mimic Library ELISA

A preliminary ELISA assay was undertaken to optimise the concentration of 28mer immobilisation (0.1-2 ng) to the 96 well plate. The results suggest that a concentration of 1.5 ng was sufficient to generate an adequate signal at 450 nm.

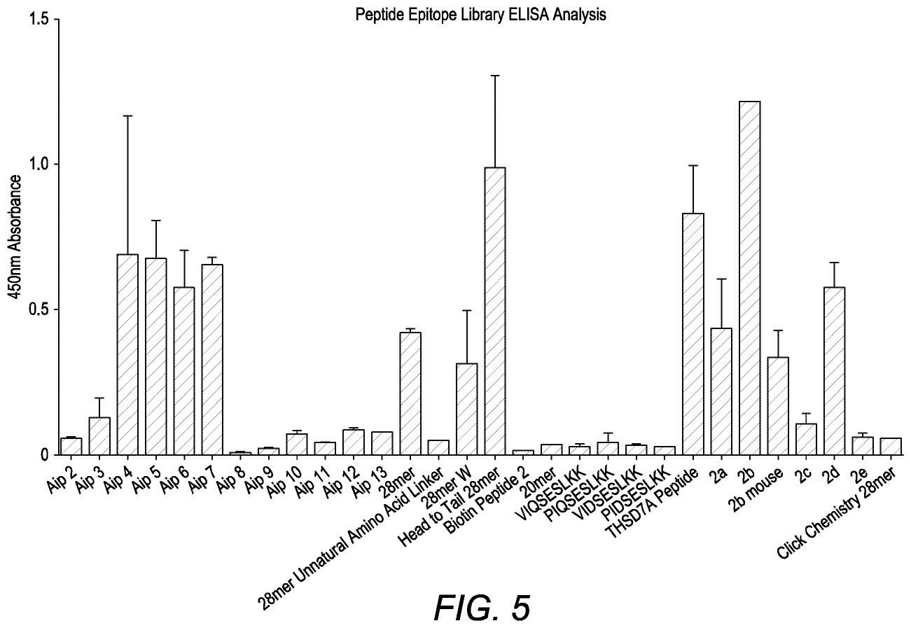

The peptide epitope library was then assessed by the optimised ELISA assay. FIG. 5, highlights that 18 peptides showed little or no association with the mouse 20 Ab in the ELISA assay. 14 of these peptides (AIP 8, 9, 10, 11, 12, 13, biotin peptide 2, 2c, 2e and PEPperPRINT peptides) also showed no interaction by slot blot analysis. AIP 2,3 Unnatural amino acid linker 28mer and click chemistry 28mer peptides, comprising modified cyclic regions, interacted with the mouse 20 in the slot blot analysis but showed no interaction in the ELISA assay. Eight peptides which had previously shown no interaction with the mouse 20 Ab, in the slot blot analysis, where found to interact with the mouse 20 Ab in the ELISA assay (AIP 4, 5, 6, 7, 2a, 2b, 2b mouse and 2d). All eight peptides contain Peptide 2 amino acid sequence (SVLTLENCK (SEQ ID NO: 35)). Four peptides (28mer (SEQ ID NO: 22), 28mer W, head to tail 28mer (SEQ ID NO: 74) and THSD7A peptide (SEQ ID NO: 91) displayed association with mouse 20 Ab in both the ELISA and slot blot analyses.

High variability in peptide binding with mouse 20 Ab between the two techniques with approximately >50% of the peptide library, including the PEPperPRINT peptides, showing little to no association in either test. It currently is not clear which technique is the most accurate and therefore three of the high preforming peptides in both techniques (28mer (SEQ ID NO: 22), head to tail 28mer (SEQ ID NO: 74) and THSD7A peptide (SEQ ID NO: 91) were selected for further analysis. In addition, Peptide 2 (SEQ ID NO: 35) shown to be the core sequence in high performing ELISA analyses was also selected.

Candidate Peptide Mouse 20 Antibody Binding Kinetics

Three approaches to determine direct Ab binding kinetics were evaluated including Microscale thermophoresis (MST), Q-sense and ProteOn SPR. The MST approach was found to require a large volume of concentrated Ab to prepare the fluorescent tag. In addition, the short half-life of the fluorescent tags made the approach not viable. The Q-sense approach enabled real-time binding to determine peptide kinetics through Kd constants. However, issues associated with non-specific binding to the silicon dioxide chip and inability to ensure the chip is sufficiently striped of peptide from previous analyses affecting accuracy and cost. ProteOn SPR enabled the assessment of multiple peptides at once without the limitations of the other two approaches, and was therefore selected for further analysis.

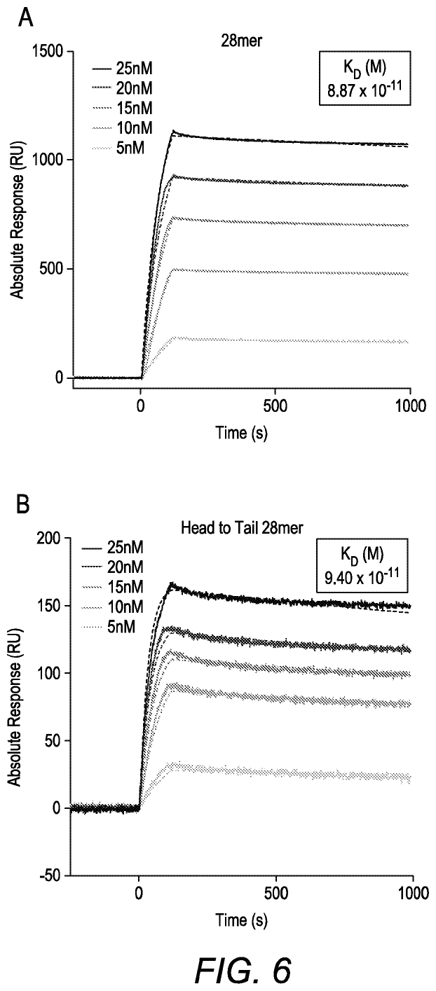

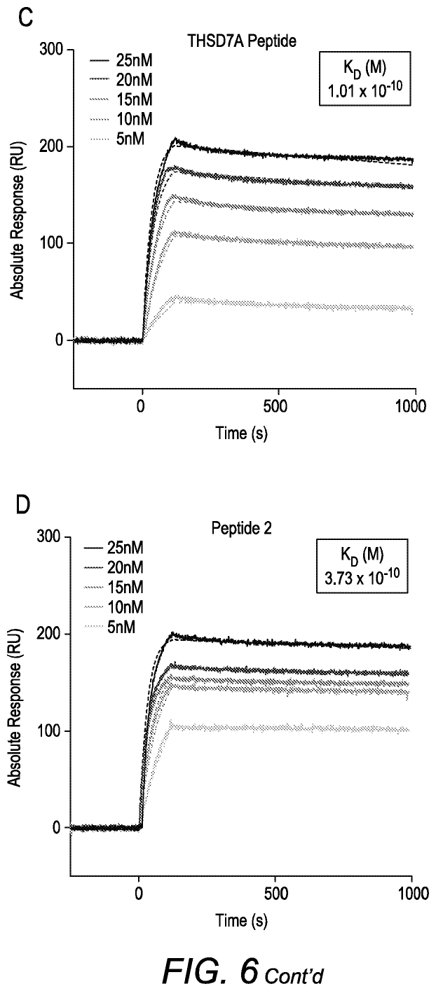

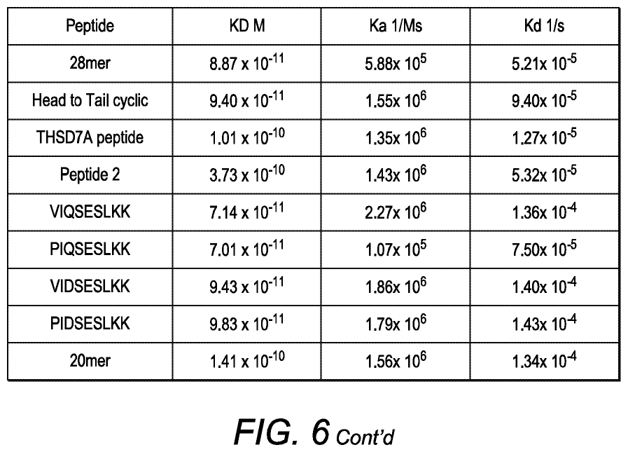

The peptides selected in 4.6.2 were evaluated by ProteOn SPR. Peptides were immobilised on a GLC chip and mouse 20 Ab injected at a concentration of 5-25 nM. The results, shown in FIG. 6, indicates that all peptides bind to Mouse 20 Ab with similar Ka, Kd values shown in Table 2. All peptides were found to have a high affinity for the mouse 20 Ab with K_{D} values ˜0.1 nM potentially suggesting the peptides have a common C-Terminal sequence (SVLTLENCK (SEQ ID NO: 35)) within the 28mer which binds to the mouse 20 Ab.

TABLE 2
Assessment of candidate peptides by anti-PLA2R mouse 20 Ab
binding kinetics

| Peptide | KD M | Ka 1/Ms | Kd 1/s |
|---|---|---|---|
| 28mer | 8.87 × 10⁻¹¹ | 5.88 × 10⁵ | 5.21 × 10⁻⁵ |
| (SEQ ID NO: 22) | | | |
| Head to Tail cyclic | 9.40 × 10⁻¹¹ | 1.55 × 10⁶ | 9.40 × 10⁻⁵ |
| (SEQ ID NO: 74) | | | |
| THSD7A peptide | 1.01 × 10⁻¹⁰ | 1.35 × 10⁶ | 1.27 × 10⁻⁵ |
| (SEQ ID NO: 91) | | | |
| Peptide 2 | 3.73 × 10⁻¹⁰ | 1.43 × 10⁶ | 5.32 × 10⁻⁵ |
| (SEQ ID NO: 35) | | | |
| VIQSESLKK | 7.14 × 10⁻¹¹ | 2.27 × 10⁶ | 1.36 × 10⁻⁴ |
| (SEQ ID NO: 20) | | | |
| PIQSESLKK | 7.01 × 10⁻¹¹ | 1.07 × 10⁵ | 7.50 × 10⁻⁵ |
| (SEQ ID NO: 4) | | | |
| VIDSESLKK | 9.43 × 10⁻¹¹ | 1.86 × 10⁶ | 1 40 × 10⁻⁴ |
| (SEQ ID NO: 18) | | | |
| PIDSESLKK | 9.83 × 10⁻¹¹ | 1.79 × 10⁶ | 1.43 × 10⁻⁴ |
| (SEQ ID NO: 19) | | | |
| 20mer | 1.41 × 10⁻¹⁰ | 1.56 × 10⁶ | 1.34 × 10⁻⁴ |
| (SEQ ID NO: 25) | | | |

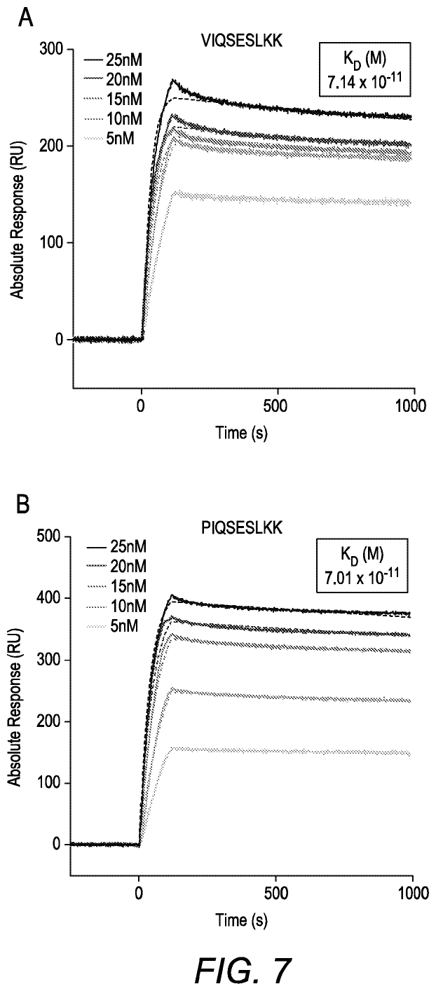

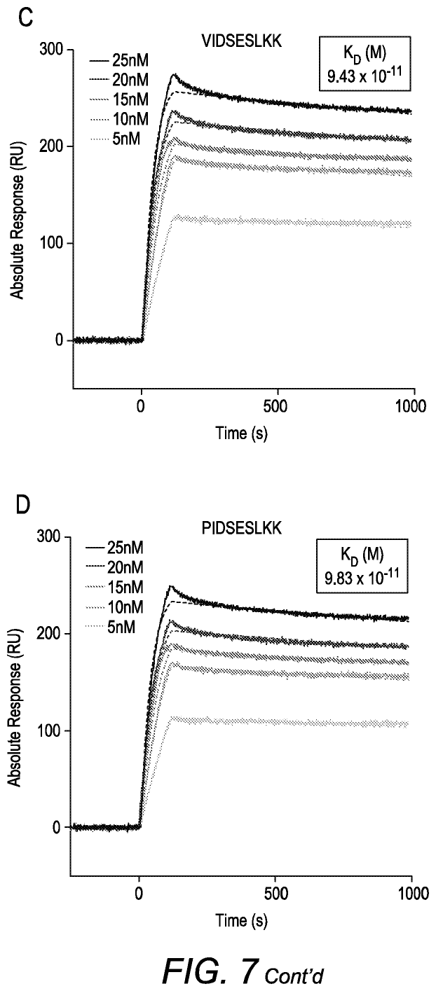

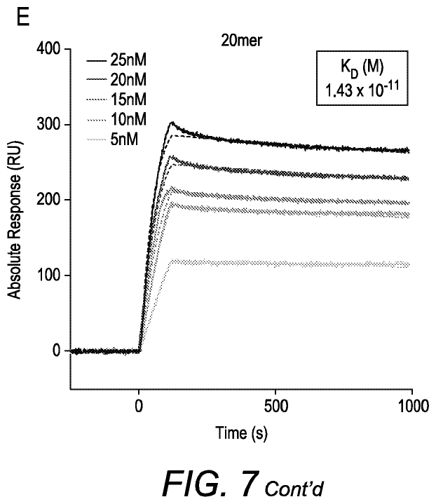

The ProteOn SPR approach was used to validate ELISA and slot blot peptide screening evaluation of PEPperPRINT peptides. The SPR analysis indicates that all PEPperPRINT peptides bind to mouse 20 Ab with similar Ka, Kd and with all KD values being ˜0.1 nM shown in Table 2, (FIG. 7). The PEPperPRINT peptide kinetics appears to be extremely similar to the peptides assessed in FIG. 6. These findings potentially indicate that all peptides have a common N-Terminal sequence (VIQSES (SEQ ID NO: 9)) which is involved binding to the mouse 20 Ab. Negative controls of anti-PLA2R rabbit Ab showed no binding to any of the peptides assessed in FIGS. 6 and 7 confirming previous slot blot evaluation of the 28mer and 31mer peptides.

Peptide Sequence Optimisation Phase 2

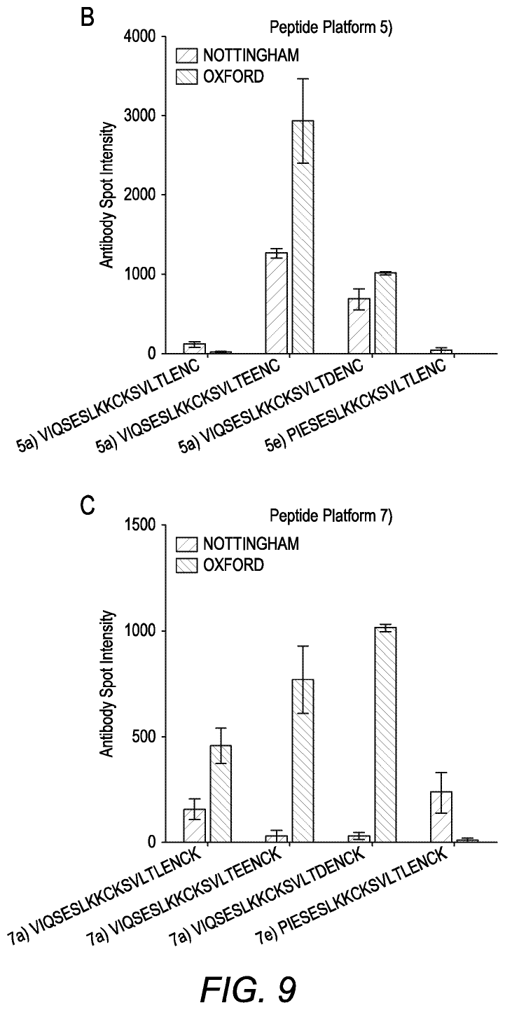

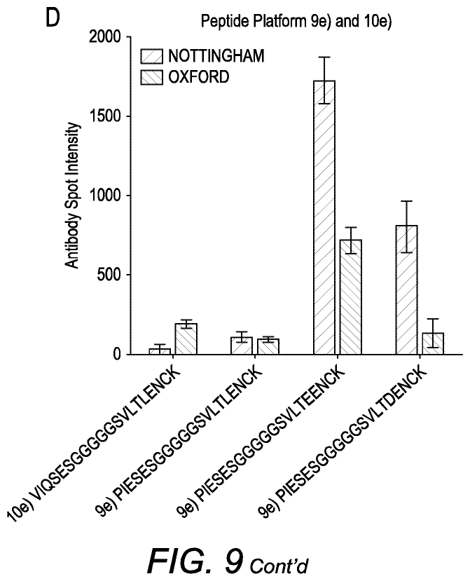

The second phase of peptide sequence optimisation had three core objectives. The first objective was to determine the key amino acid components of the 28mer cyclic region and beneficial mutations for binding with human autoAbs. The second objective was to understand the impact of the beneficial mutations identified in the N-Terminus on the cyclic region. The third objective was to assess whether joining N-Terminus (VIQSES (SEQ ID NO: 9)) and C-terminus regions via an intervening glycine spacer (1-5 amino acids) maintains or enhances human autoAb binding. PEPperPRINT® were assigned to explore these objectives using their peptide microarray incorporating a 1 in 10 dilution of three patient sera (Nottingham, Dundee and Oxford; FIG. 9).

The following peptide optimisation platforms were designed to explore objectives one and two: peptide platform 5, cyclic region A (Peptide 2, CKSVLTLENC (SEQ ID NO: 26)); peptide platform 6, cyclic region B (without Peptide 2 CIAGKLENC (SEQ ID NO: 27)); peptide platform 7, C cyclic region A incorporating the 28mer's N-Terminal lysine, and peptide platform 8, cyclic region B incorporating the 28mers N-Terminal lysine. Within each peptide platform the following N-terminus linear amino acid sequences were attached to the corresponding cyclic regions (a VIQSESLKKCK (SEQ ID NO: 28), b PIQSESLKKCK (SEQ ID NO: 29), c VIESESLKKCK (SEQ ID NO: 30), d VIDSESLKKCK (SEQ ID NO: 31), e PIESESLKKCK (SEQ ID NO: 32) to assess the impact of previously identified beneficial mutations. A substitution scan between the two cysteine residues (from either cyclic region A; CKSVLTLENC (SEQ ID NO: 26) or B; CIAGKLENC (SEQ ID NO: 27)) were undertaken on peptide platforms 5-8 part a. Two additional peptide platforms comprising potential N-Terminus sequences (peptide platform 9 PIESES (SEQ ID NO: 11) or peptide platform 10 VIQSES (SEQ ID NO: 9)) by intervening, different length, glycine spacers (a G; b GG, c GGG, d GGGG (SEQ ID NO: 33), e GGGGG (SEQ ID NO: 34)) to Peptide 2 (SVLTLENCK (SEQ ID NO: 35)). Peptide platform 9e included a substitution scan within the Peptide 2 sequence (SVLTLENC (SEQ ID NO: 21)) FIG. 8.

Overall only peptide platforms containing Peptide 2 (5, 7, 9 and 10e) and peptide platform 11, (FIG. 8) were found to exhibit high autoAb binding with patient sera from Nottingham and Oxford FIGS. 9 B, C and D). Experiments with the Dundee patient serum showed only background noise levels of interaction.

The wild type sequence peptide platform 5a showed relatively low autoAb binding in both the Nottingham (˜112 A.U.) and Oxford (˜17 A.U.) sera. However, the inclusion of certain mutations within the Peptide 2 region was found to significantly increase autoAb binding. Specifically, substitution of leucine at position 24 with either glutamate or aspartate generate increased the magnitude of autoAb binding in the Nottingham serum (˜1257 and ˜673 A.U. respectively) and to an even greater extent in the Oxford serum (˜2939 and 1011 A.U. respectively). Changing the wild type N-Terminus (peptide platform 5 a) to include a PIESES (SEQ ID NO: 11) sequence reduced binding intensity in both patient sera tested (Nottingham ˜37 A.U. and Oxford ˜0 A.U). Mutations in the N-Terminus (peptide platforms 5 b, c and d) previously shown to be beneficial in 4.4 made no impact on binding intensity in both patient sera.

Peptide platform 7, which was similar to peptide platform 5 but included an N-Terminal lysine at position 28, showed that the wild type (peptide platform 7a) produced equivalent binding intensity to peptide platform 5a wild type in the Nottingham serum (˜115 A.U.). However, in the Oxford sample the binding intensity was significantly higher (˜456 A.U.). Similarly, substitution of leucine at position 24 by glutamate and aspartate produced increased in autoAb binding (˜768 and 1011 A.U. respectively) in the Oxford serum. The level of response for the glutamate mutation was approximately three fold lower than observed in peptide platform 5a. Whereas the level of binding for the aspartate mutation in both platforms was the same. In contrast, glutamate and aspartate mutations challenged with the Nottingham patient sera showed reduced antibody binding compared to the wild type (˜27 and ˜29 A.U.). These results potentially indicate that the combination of either mutation with the C-terminal lysine has a detrimental effect on autoAb binding particularly in the Nottingham sample. Changing the wild type N-Terminus (peptide platform 7a) to include a PIESES (SEQ ID NO: 11) sequence increased binding intensity in the Nottingham sample (˜235 A.U.) whilst binding in the Oxford (˜8 A.U.) sample was reduced, however, the magnitude of change is potentially too small to draw conclusions.

Only peptide platforms 9e and 10e including glycine spacers showed significant improvement in autoAb binding in both the Nottingham (peptide platform 9e ˜109 A.U and 10e ˜33 A.U.) and Oxford samples (9e ˜95 A.U, 10e ˜195 A.U.). Yet again, substitution of leucine at position 24 with either glutamate or aspartate increased the level of autoAb binding in the Nottingham serum (˜1730 and ˜721 A.U. respectively) and, to a lesser extent in the Oxford serum (˜806 and 130 A.U. respectively).

It has been shown that there is a link between anti-PLA2R autoAb titre, disease progression and severity in patients with IMN. Existing immunosuppressive therapies are known reduce the levels of anti-PLA2R autoAb titre but are highly toxic limiting their use. A peptide epitope mimic which could outcompete the binding of autoAbs to PLA2R, potentially alleviating symptoms of IMN paving the way for a new form of peptide therapeutics (PT). Here we assess and optimise candidate peptides solubility ahead.

Semi quantitative pre-screening of peptides by slot blot and ELISA identified four peptides (28mer (SEQ ID NO: 22), 28mer W, head to tail 28mer (SEQ ID NO: 74) and THSD7A peptide (SEQ ID NO: 91) that associated with the mouse 20 Ab. Three of the peptides are derivatives of the 28mer indicating that binding appears to be unaffected by subtle sequence differences. The core sequence in Peptide 2 (SVLTLENCK (SEQ ID NO: 35)) was present in all four proteins albeit slightly modified in the THSD7A peptide sequence. Alternative chemistry for cyclising the 28mer did not lead to successful binding in the ELISA assay suggesting that natural cyclisation may be important to autoAb binding. 10 peptides which showed binding to the mouse 20 Ab had the Peptide 2 amino acid sequence (SVLTLENCK (SEQ ID NO: 35)) in common, suggesting that this is a key sequence.

High variability in peptide binding with mouse 20 Ab was observed between the two techniques with 30% of peptides tested showed different binding results between assays. Both techniques have limitations. For example, peptides that are smaller than 0.2 microns may pass through the slot blot membrane leading to false negatives. Additionally, peptides may aggregate during the ELISA assay potentially blocking critical amino acids from binding with the Ab, again leading to false negatives. Both techniques assess peptides immobilised to surfaces and not how they behave in solution. Overall, >50% of the peptide library, including the 20mer (SEQ ID NO: 25) which was shown to bind to the autoAb in the optimisation analysis, showed little to no association in either test reinforcing the need for multiple techniques to assess Ab binding interaction.

Direct binding kinetics for the 28mer (SEQ ID NO: 22), head to tail 28mer (SEQ ID NO: 74), THSD7A peptide (SEQ ID NO: 91) and Peptide 2 (SEQ ID NO: 35) with mouse 20 Ab indicated all four peptides tightly bound to the Ab with KD values of ˜0.1 nM, suggesting they have a common sequence that is important for Ab binding. Peptide 2 (SEQ ID NO: 35) is the only common region in all four peptides suggesting that this sequence is involved in Ab binding. Unexpectedly, peptides that were specifically designed to test the impact of beneficial amino acid substitutions identified in the first phase of peptide optimisation (VIQSESLKK (SEQ ID NO: 20), PIQSESLKK (SEQ ID NO: 4), VIDSESLKK (SEQ ID NO: 18), PIDSESLKK (SEQ ID NO: 19) and the 20mer also bound tightly to the mouse 20 Ab (KD ˜0.1 nM). The peptides (VIQSESLKK (SEQ ID NO: 20), PIQSESLKK (SEQ ID NO: 4), VIDSESLKK (SEQ ID NO: 18), PIDSESLKK (SEQ ID NO: 19) do not contain a Peptide 2 sequence (SEQ ID NO: 35) suggesting there is potentially a second region (VIQSESLKK (SEQ ID NO: 20)) that is important for binding with the mouse 20 Ab. Additionally, beneficial mutations (PIQSESLKK (SEQ ID NO: 4), VIDSESLKK (SEQ ID NO: 18), PIDSESLKK (SEQ ID NO: 19) did not deliver improvements in mouse 20 Ab binding compared to the wild type peptide (VIQSESLKK (SEQ ID NO: 20)). This finding could indicate that the beneficial mutations may be Nottingham patient autoAb sera specific reinforcing the need to test with a broader patient sera library.

These findings suggest that the 28mer (SEQ ID NO: 22) contains two important regions for binding to the mouse 20 Ab at the N (VIQSESLKK (SEQ ID NO: 20)) and C (SVLTLENCK (SEQ ID NO: 35)) terminal regions. This observation implies that the N and C terminal regions make up the epitope that is recognised by the mouse 20 Ab. A potential limitation to these observations is that human autoAbs may respond differently either recognising only one region or requiring both sequences in combination.

The second phase of peptide optimisation of the 28mer sequence (SEQ ID NO: 22) indicated that cyclic region B (CIAGKLENC (SEQ ID NO: 27)) was unable to bind to the Ab even though the linear peptide (VIQSES (SEQ ID NO: 9)) with and without N-terminal mutations were present. These finding provides further evidence for the importance of the peptide 2 sequence (SEQ ID NO: 35) and also indicates that the linear region alone is insufficient to enable binding. Leucine (position 24) substitution with either glutamate or aspartate was again found to increase the level of autoAb binding, providing additional evidence that increasing the negative charge at this point in the sequence is beneficial in both sera. Additionally, presence of C-Terminal lysine appears to be neutral and, in some cases, deleterious to Ab binding potentially by neutralising the negative charge imparted by other beneficial mutations. Glycine spacer peptides (5 glycine amino acids) with no cyclic confirmation appear to be equivalent to cyclic peptides suggesting steric distance between epitopes is an important factor rather than a requirement for cyclic conformation. This study further highlights evidence of patient autoAb sera specific binding preferences. For example, the Oxford sample showed increased affinity for VIQSES (SEQ ID NO: 9) N-Terminal sequence whereas the Nottingham sample showed increased affinity for the PIQSES N-Terminal sequence. In contrast, the Dundee patient serum showed only background noise levels of interaction suggesting the serum either didn't recognise any of the peptide platforms tested or was no longer viable. Variability between patient sera autoAbs is a major limitation for determining optimised peptide sequences reinforcing the need to assess binding affinity across a large patient sera library. However, this is also potentially a very exciting finding from a disease understanding perspective which may provide insight into disease progression, potentially allowing us to identify epitopes linked to disease severity. A further limitation could be that immobilisation of the peptide to the microarray may artificially create the observed requirement for both the N and C terminal regions for binding to take place. Consequently, assays conducted in a fluid environment are required to validate these findings.

Solubility analyses have identified that time in solution had a major impact on the solubility of the 31mer peptide, which could not be improved by modifying the buffer environment. Further studies showed that the 28mer (SEQ ID NO: 22) had inherently superior solubility characteristics, with 85% peptide remain in solution over 24 hours. In addition, subtle changes in the peptide sequence, in this case removal of three amino acids and insertion of tryptophan at position 24, had a dramatic impact on peptide solubility. Further solubility improvements can be achieved by optimising the test tube composition as demonstrated by Goebel-Stengel et al study (Goebel-Stengel et al., 2011a). In this study siliconised and LoBind tube materials provided a further 10% improvement in solubility. Importantly Goebel-Stengel et al noted the lack of consensus between tube materials and the solubility of different peptides. Consequently, potential candidate peptides solubility will need to be assessed and test tube environment optimised to ensure a soluble active peptide is ultimately selected.

In summary, my work has identified the critical amino acids (VIQSES (SEQ ID NO: 9) and SVLTLENC (SEQ ID NO: 21) within the 28mer peptide sequence (SEQ ID NO: 22) that are important for binding to anti-PLA2R autoAbs. Additionally, this study has demonstrated that it may be possible to replace the cyclic structure with a 5 glycine spacer. A 19amino acid sequence comprising VIQSES (SEQ ID NO: 9) and SVLTLENC (SEQ ID NO: 21) linked by a 5 glycine spacer may provide the optimal balance of Ab binding, peptide stability and scalable manufacture. However, further validation including screening with human IMN anti-PLA2R autoAb patient sera library is required. This study has built a strong platform to base future evaluations of candidate peptide functional characteristics in vitro and in vivo.

Surface Photon Resonance Inhibition Assay (SPR).

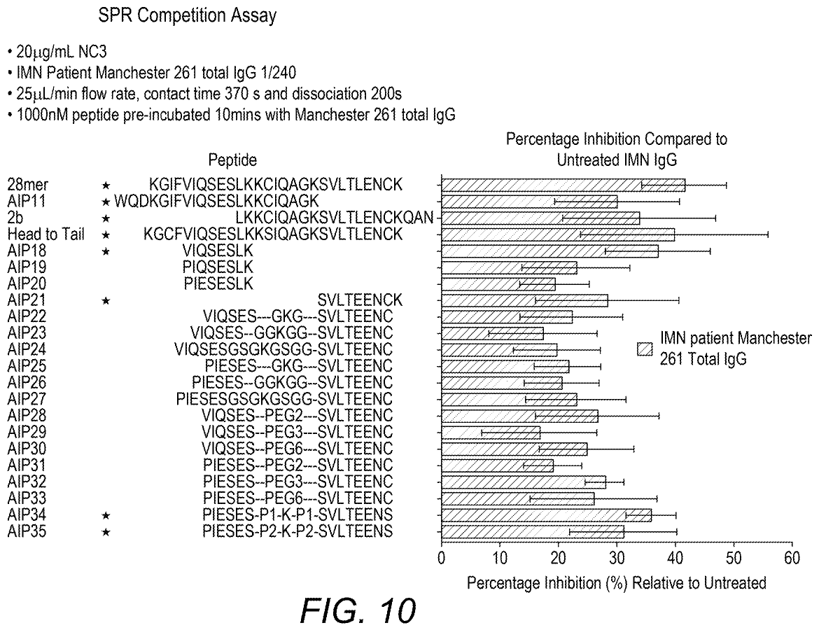

NC3 protein at 20 ug/ml was coated on the chip. A 1/240 dilution of Anti-PLA2R IgG (Manchester 261) was incubated separately with aliquots of the peptides listed in FIG. 10 at 1 uM concentration. After a preincubation of antibody and peptide for 10 minutes, each solution was flowed over the chip to allow binding of the anti-PLA2R to the chip surface. Each peptide-antibody solution was subjected to a standard set of conditions of flow rate, contact time and dissociation time. The amount of anti-PLA2R bound to the NC3 on the chip surface was measured and expressed as a percentage of the control anti-PLA2R solution containing no peptides. Comparing the ability of the peptides listed in FIG. 10 to bind to the anti-PLA2R and thus inhibit the anti-PLA2R from binding to the NC3 fragment of PLA2R on the chip surface.

The native 28mer peptide sequence illustrates the maximum inhibition of anti-PLA2R binding (40%) to be expected under the conditions of this assay. FIG. 10 clearly demonstrates that the synthetic peptide sequences of the invention show similar abilities to bind to anti-PLA2R in solution and inhibit (from 20-40%) the antibody from binding to the chip surface coated with NC3 PLA2R.

### REFERENCES

AUCAN, C., TRAORE, Y., TALL, F., NACRO, B., TRAORE-LEROUX, T., FUMOUX, F. & RIHET, P. 2000. High immunoglobulin G2 (IgG2) and low IgG4 levels are associated with human resistance to Plasmodium falciparum malaria. Infect Immun, 68, 1252-8.
DAVIES, A. M., RISPENS, T., OOIJEVAAR-DE HEER, P., GOULD, H. J., JEFFERIS, R., AALBERSE, R. C. & SUTTON, B. J. 2014. Structural determinants of unique properties of human IgG4-Fc. J Mol Biol, 426, 630-44.
SCHINDELIN et al. 2012

1. A non-naturally occurring peptide comprising amino acid sequence S-V-L-T-X1-E-N-X2 (SEQ ID NO: 1), wherein X1 is any amino acid and X2 is any amino acid.

2. The peptide according to claim 1, wherein the peptide is a variant peptide comprising the amino acid sequence SVLTEENC (SEQ ID NO: 2) or SVLTEENS (SEQ ID NO: 3).

3. The peptide of claim 1, wherein the peptide comprises:
A C-terminal domain comprising the amino acid sequence S-V-L-T-X1-E-N-X2 (SEQ ID NO: 1)
An N-terminal domain comprising the amino acid sequence X3-I-X4-X5-E-X6 (SEQ ID NO: 5), wherein, X3, X4, X5 and X6 each denote any amino acid.

4. The peptide according to claim 3, wherein X3 is V or P; X4 is Q, D or E; X5 is S, D or E and X6 is S, D or E.

5. The peptide according to claim 1, wherein the peptide is a variant peptide comprising the amino acid sequence S-V-L-T-X1-E-N-X2-K (SEQ ID NO: 6).

6. The peptide of claim 3, wherein the N-terminal domain comprises the variant peptide comprising the amino acid sequence X3-I-X4-X5-E-X6-L-K (SEQ ID NO: 8).

7. The peptide according to claim 3, wherein the peptide comprises a linker between the N-terminal and C-terminal domains.

8. The peptide according to claim 7, wherein the linker is selected from a group consisting of: a peptide linker, a synthetic linker, a combination of peptide and synthetic linker.

9. (canceled)

10. The peptide according to claim 8, wherein the peptide linker comprises 5 glycine residues, the synthetic linker comprises a PEG molecule.

11. The peptide according to claim 8, wherein the synthetic linker comprises a PEG molecule.

12. (canceled)

13. The peptide according to claim 1, wherein the number of amino acid residues in the peptide is less than or equal to 28 amino acid residues.

14. (canceled)

15. The peptide according to claim 1, wherein X1 is L or E and X2 is C or S.

16. The peptide according to claim 3, wherein the N-terminal domain comprises a variant of the sequence VIQSES (SEQ ID NO: 9) such that one or more of the following substitutions are made: V to P, Q to D or E, optionally either or both S to D or E.

17. The peptide according to claim 3, wherein the N-terminal domain comprises the sequence PIDDES (SEQ ID NO: 10) or PIESES (SEQ ID NO: 11).

18. The peptide according to claim 3, wherein the peptide comprises the amino acid sequence X3-I-X4-X5-E-X6-PEG-K-PEG-S-V-L-T-X1-E-N-X2 (SEQ ID NO: 12).

19. The peptide according to claim 1, wherein the peptide comprises PIESES-PEG-K-PEG-SVLTEENC (SEQ ID NO: 13); VIQSES-PEG-K-PEG-SVLTLENC (SEQ ID NO: 14); VIQSES-PEG-K-PEG-SVLTEENC (SEQ ID NO: 15); PIDDES-PEG-K-PEG-SVLTLENC (SEQ ID NO: 16); PIDDES-PEG-K-PEG-SVLTEENC (SEQ ID NO: 17).

20. (canceled)

21. A pharmaceutical composition comprising a peptide according to claim 1 and a pharmaceutically acceptable carrier.

22-32. (canceled)

33. A method of preventing or treating kidney disease in a subject, the method comprising:
contacting a volume of the subject's blood with at least 1 peptide according to claim 1, such that anti-PLA2R antibodies present in the subject's blood are able to bind to and be retained by the peptide; and
separating the peptide and bound anti-PLA2R antibody from the blood, to yield an antibody-depleted volume of blood.

34. The method according to claim 33, wherein a batch of blood comprising one or more volumes of blood to be treated, is removed from the subject, and the steps of contacting a volume of the blood with at least 1 peptide, and subsequent separation of the peptide and bound antibodies, completed to yield a batch comprising the volume, or volumes, of antibody-depleted blood.

35. The method according to claim 33, wherein the steps of contacting the blood with the binding partner, and subsequent separation, are carried out “in line” wherein the binding partner is provided in an arrangement so that the patient's blood may flow over the binding partner, thus allowing it to bind anti-PLA2R antibodies in the blood.

36-59. (canceled)
